(19) United States
(12) Patent Application Publication
(10) Pub. No.: US 2011/0112032 A1
(43) Pub. Date: May 12, 2011

(54) Regulation of Brain Natriuretic Peptide and Catecholamines for the Treatment of Cardiovascular Diseases

(76) Inventors: Ming-He Huang, League City, TX (US); Barry Uretsky, Fort Smith, AR (US)

(21) Appl. No.: 11/991,866

(22) PCT Filed: Sep. 12, 2006
(86) PCT No.: PCT/US2006/035564

§ 371 Date: Jan. 10, 2011

Related U.S. Application Data
(60) Provisional application No. 60/716,324, filed on Sep. 12, 2005.

(51) Int. Cl. A61K 38/00; A61K 31/4422; A61P 25/00; A61P 9/00; A61P 9/04

(52) U.S. Cl. 514/16.4; 514/17.7; 514/356

(57) ABSTRACT

The present invention describes methods for treating heart diseases by pharmacological manipulation of intrinsic cardiac adrenergic cells. The activation or inhibition of δ-opioid receptors or selective β2-adrenergic receptor agonist in these cells can help regulate catecholamine synthesis and release in these cells. Thus drugs directed towards these receptors can be used in the treatment of heart diseases including acute and chronic congestive heart failure, as well as acute and chronic coronary artery disease. The present invention also describes a method of treating acute decompensated congestive heart failure using δ-opioid receptor agonists or selective β2-adrenergic receptor agonists to stimulate endogenous production of brain natriuretic peptide. This invention also provides a novel strategy for protection against myocardial ischemia through the specific activation of δ-opioid receptors or selective β2-adrenergic receptor agonists exclusively expressed by intrinsic cardiac adrenergic cells in human heart. The activation of intrinsic cardiac adrenergic cells by δ-opioid receptor stimulation enhances endogenous catecholamine release, which subsequently stimulates myocardial alpha 1-adrenoreceptors achieving myocardial protection in patients suffering from myocardial ischemia due to coronary artery disease.

### CROSS-REFERENCE TO RELATED APPLICATIONS

This U.S. national stage application is filed under 35 U.S.C. §363 and claims benefit of priority under 35 U.S.C. §365 of international application PCT/US2006/035564, filed Sep. 12, 2006, now abandoned, which claims benefit of priority under 35 U.S.C. §119(e) of provisional application U.S. Ser. No. 60/716,324, filed Sep. 12, 2005, now abandoned.

### BACKGROUND OF THE INVENTION

1. Field of the Invention

The present invention relates generally to the field of cardiology. More specifically the invention relates to the endogenous release of brain natriuretic peptide (BNP) and regulation of catecholamines by pharmacological manipulation of delta-opioid receptor expressed by intrinsic cardiac adrenergic (ICA) cells for the treatment of cardiovascular diseases.

2. Description of the Related Art

Brain natriuretic peptide has important roles in the regulation of cardiovascular function. Brain natriuretic peptide has cardiovascular beneficial effects, including peripheral and coronary vasodilation, natriuresis, inhibition of renin-angiotenin-aldosterone axis (VMAC Investigators, 2002), and inhibition of myocardial fibrosis (De Lemos and Morrow, 2002). Brain natriuretic peptide exerts an important compensatory role in sustaining cardiac output in decompensated congestive heart failure (CHF). Great elevation of plasma brain natriuretic peptide is observed in patients with acute congestive heart failure suggesting a compensatory mechanism of brain natriuretic peptide release in heart failure (Maisel et al, 2002). Clinical trials have demonstrated that intravenous administration of a recombinant human brain natriuretic peptide, nesiritide, to patients with decompensated congestive heart failure dramatically reduces pulmonary capillary wedge pressure, improves symptoms, and allows quicker hospital discharge (Colucci et al, 2000). In patients with congestive heart failure due to isolated diastolic dysfunction, infusion of brain natriuretic peptide reduces left atrial pressure during exercise (Clarkson, 1996). Despite the clinical success in utilizing intravenous brain natriuretic peptide infusion for congestive heart failure treatment, the major limitation is its prohibitive cost and the need to administer it as a continuous intravenous infusion, which requires extended hospitalization of a patient requiring the treatment. Exogenous brain natriuretic peptide is ineffective when taken orally because the peptide is degraded during digestion.

Basic knowledge regarding the origin, synthesis and release of brain natriuretic peptide is not complete. For example, there is no data that establishes that cardiac cells synthesize brain natriuretic peptide. Without such knowledge one cannot exclude the possibility that brain natriuretic peptide is simply taken up and stored by myocytes, after its release from another cell type. In terms of brain natriuretic peptide metabolism, there is little information available concerning how brain natriuretic peptide release is regulated. Initially, ventricular stretch was thought to stimulate brain natriuretic peptide release. Recent data has shown that hypoxia in the absence of ventricular stretch can also cause release of brain natriuretic peptide. This indicates that chemical stimulation may provide a means of releasing brain natriuretic peptide. Another potential mechanism to increase brain natriuretic peptide level in plasma is reduced brain natriuretic peptide degradation. The only potential drug that elevates circulating brain natriuretic peptide levels is a neutral endopeptidase inhibitor, CANDOXATRIL, that inhibits brain natriuretic peptide degradation (Lang et al. 1991). However CANDOXATRIL has many vasoactive substrates like angiotensin II, endothelin and bradykinin (Kentsch, 1999), and so using it to increase brain natriuretic peptide levels in plasma is not desirable. Thus, there is a need to understand regulatory mechanisms of brain natriuretic peptide synthesis and release and thereby develop cost effective pharmaceutical agents to mobilize endogenous brain natriuretic peptide release.

The American College of Cardiology AMERICAN COLLEGE OF CARDIOLOGY™/AMERICAN HEART ASSOCIATION© heart failure guidelines include morphine as a first line of treatment for acute heart failure. The mechanism underlying morphine-mediated symptomatic relief is not known. Long-term opiate exposure mitigates coronary artery disease severity and its fatal consequences in opiate users. This is indicative of the presence of opiate receptors in the heart.

Intrinsic cardiac adrenergic cells are cardiac neuroendocrine cells that express genes and enzyme proteins required for catecholamine biosynthesis (Huang et al, 1996). Intrinsic cardiac adrenergic cells generate spontaneous [Ca²⁺]_{I} transients through a calcium influx mechanism. The activity of intrinsic cardiac adrenergic cells is enhanced following hypoxial/reoxygenation stimulation and suppressed by L-type calcium channel blocker (Huang et al, 2003). The identification of spontaneous [Ca²⁺]_{I} transients generated by intrinsic cardiac adrenergic cells provide a physiological basis for constitutive neurotransmitter release by these cells. Opiate mediated cardioprotection in ischemia is related to the release of endogenous epinephrine by cardiac cells. The presence of catecholamines and [Ca²⁺]_{I} transients in intrinsic cardiac adrenergic cells indicate this release of epinephrine in the presence of an opiate may occur from intrinsic cardiac adrenergic cells. Catecholamines are well known to provide cardioprotection against ischemia. Activation of myocardial alpha-adrenergic and, to some extent, beta-adrenergic receptors provides powerful infarct size reduction and improved functional recovery following myocardial infarction (Bankwala et al, 1994; Cleveland et al, 1997; Kitakaze et al, 1994). Recent studies indicate that δ-opioid receptor (DOR) agonists confer a similar degree of cardioprotection against ischemia (Bell et al, 2000; Guo et al, 2005; Schultz et al, 1998; Sigg et al, 2002). Both adrenergic and δ-opioid stimulation mimic ischemic preconditioning. Interestingly, cardioprotection associated with adrenergic and δ-opioid stimulation utilize the same final signaling pathways involving protein kinase C and ATP-sensitive K⁺ channels (Fryer et al, 1998; Fryer et al, 2001; Ito et al, 2001; Kaku et al, 1991; Huang et al, 2005). Although d-opioid receptor agonists exert cardioprotection, it is unclear which type of heart cell expressing d-opioid receptor mediates the effect.

Currently there are 5 million Americans with congestive heart failure, with nearly 500,000 new cases every year. The current treatment involves use of synthetic catecholamines (mainly dopamine and dobutamine) and recombinant brain natriuretic peptide. These drugs must be infused intravenously in a coronary care unit setting with very high costs. Further, the safety of exogenous infusion of dobutamine and brain natriuretic peptide could be a concern since they may increase mortality due to some unknown factors. Thus strategies targeting endogenous catecholamines and brain natriuretic peptide mobilization are attractive and more cost effective alternative in treating heart failure.

Despite this, the prior art is lacking in means for endogenous regulation of catecholamines and brain natriuretic peptide in intrinsic cardiac adrenergic cells to combat heart failure. The present invention fulfills this longstanding need and desire in the art.

### SUMMARY OF THE INVENTION

In one embodiment, the present invention is directed to a method for endogenous release of brain natriuretic peptide and catecholamines from intrinsic cardiac adrenergic cells to treat cardiovascular disease. The excessive release of brain natriuretic peptide in one embodiment can be mediated by agonists of the δ-opioid receptors exclusively present in intrinsic cardiac adrenergic cells.

In another embodiment, the present invention is directed to a method for up-regulating catecholamine synthesis and release from intrinsic cardiac adrenergic cells by stimulating the δ-opioid receptors present in these cells. The method in this embodiment can be applied to provide neurohormonal support to the heart of an individual suffering from decompensated heart failure through endogenous adrenergic stimulation of the heart with catecholamines like epinephrine and, concomitantly, brain natriuretic peptide. Activation of protein kinase A and L-type calcium channels is required to up-regulate catecholamine synthesis and release from the intrinsic cardiac adrenergic cells via δ-opioid receptors. Thus agonists of δ-opioid receptors, for example [D-Pen²⁵]-enkephalin (DPDPE), can be used to increase [Ca²⁺]_{I} transients to activate release of catecholamines from intrinsic cardiac adrenergic cells. Agents that activate protein kinase A can also activate the δ-opioid receptor pathway for up-regulation of catecholamines and brain natriuretic peptide.

In yet another embodiment, the present invention also discloses a method for down-regulating synthesis and release of catecholamines to prevent excessive adrenergic stimulation of the heart in an individual suffering from chronic congestive cardiac failure. Antagonists of intrinsic cardiac adrenergic cell δ-opioid receptors can be used to inhibit the release of catecholamines. A calcium channel blocker may be used to decrease [Ca²⁺]_{I} transients such that the release of catecholamines by intrinsic cardiac adrenergic cells is blocked. Inhibitors of protein kinase A can also be used to block the δ-opioid receptor mediated release of catecholamines by intrinsic cardiac adrenergic cells in a chronic congestive heart failure state.

In still yet another embodiment the invention presents a method for treating an individual with a disease such as cirrhosis, characterized by excessive fluid retention. Stimulation of intrinsic cardiac adrenergic cells to release endogenous brain natriuretic peptide will produce diuresis and help in the management of such diseases. In this embodiment the present invention can be practiced using a compound that stimulates δ-opioid receptors present in intrinsic cardiac adrenergic cells to synthesize and release brain natriuretic peptide.

In still yet another embodiment of the present invention, there is a method of inducing cardioprotection in an individual in need of such treatment. Such a method comprises administering a pharmacologically effective dose of a compound that activates intrinsic cardiac adrenergic cells.

### BRIEF DESCRIPTION OF THE DRAWINGS

The appended drawings have been included herein so that the above-recited features, advantages, and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1E show the co-expression of brain natriuretic peptide (FIG. 1A) and tyrosine hydroxylase (FIG. 1B) in rat ventricular tissue. Background myocytes have barely detectable brain natriuretic peptide signal (FIG. 1C). The corresponding nuclei (blue, DAPI) of intrinsic cardiac adrenergic cells and myocytes in FIG. 1C are identified in FIG. 1D. FIG. 1E illustrates the localization of brain natriuretic peptide mRNA to intrinsic cardiac adrenergic cells but not myocytes. FIG. 1F shows the nuclei of intrinsic cardiac adrenergic cells (arrows) and adjacent myocytes of the corresponding cells shown in FIG. 1E.

FIGS. 2A-2D show the immunostaining of δ-opioid receptors in intrinsic cardiac adrenergic cells (FIG. 2A) that express tyrosine hydroxylase (FIG. 2B) in rat ventricular tissue. Insets in FIGS. 2A and 2B are magnified imaging of an intrinsic cardiac adrenergic cell expressing δ-opioid receptors and tyrosine hydroxylase respectively. FIG. 2C shows intrinsic cardiac adrenergic cells retain immunoreactivity of δ-opioid receptors in cardiac cell culture. FIG. 2D shows the nucleic of intrinsic cardiac adrenergic cells shown in FIG. 2C. Calibration bar: 20 μM.

FIG. 3 shows the presence of tyrosine hydroxylase (brown) in the cytoplasm of a large cluster of intrinsic cardiac adrenergic cells in the left ventricular myocardium of a transplanted heart. No sympathetic nerve endings containing tyrosine hydroxylase were identified in this tissue section. Calibration bar: 20 μM

FIGS. 4A-4B show non-specific and specific agonists of the opioid receptor in intrinsic cardiac adrenergic cells. FIG. 4A illustrates the enhancement of [Ca^{2+]}_{I} transients generated by intrinsic cardiac adrenergic cells in the presence of morphine which is a non-specific opioid receptor agonist. The receptor mechanism elicited by morphine is presumably mediated through the activation of δ-opioid receptors expressed in intrinsic cardiac adrenergic cells. FIG. 4B shows the δ-opioid receptor specific agonist, [D-Pen²⁵]-enkephalin, induces excitatory effect on [Ca^{2+]}_{I} transients generated by intrinsic cardiac adrenergic cells. [D-Pen²⁵]-enkephalin induces a dose-dependent increase in frequency of spontaneous [Ca²⁺]_{I} transients generated by intrinsic cardiac adrenergic cells in cardiac cell culture.

FIGS. 5A-5H show the immunoreactivity of tyrosine hydroxylase (TH) and phenylethanolamine N-methyltransferase (PNMT) in intrinsic cardiac adrenergic cells. Tyrosine hydroxylase (FIG. 5A) and phenylethanolamine N-methyltransferase (FIG. 5C) immunoreactivities were identified in clusters of intrinsic cardiac adrenergic cells in tissue sections of fetal rat heart. FIGS. 5B and 5D show the nuclei of intrinsic cardiac adrenergic cells and of adjacent myocytes shown in FIGS. 5A and 5C. FIGS. 5E and 5G show the immunoreactivity of tyrosine hydroxylase and phenylethanolamine N-methyltransferase in intrinsic cardiac adrenergic cell-myocyte co-cultures respectively. FIGS. 5F and 5H show the nuclei of intrinsic cardiac adrenergic cells and adjacent myocytes (red arrow) shown in FIGS. 5E and 5G. Calibration bar: 20 μM.

FIG. 6 shows the presence of tyrosine hydroxylase (lane 1) and phenylethanolamine N-methyltransferase (lane 3) mRNAs in rat fetal heart (embryonic day 16, E16). Tyrosine hydroxylase (lane 2) and phenylethanolamine N-methyltransferase (lane 4) mRNA from maternal adrenal glands (AD) are used as positive controls.

FIGS. 7A-7B show myocytes. FIG. 7A shows the immunoreactivity of myosin heavy chain striations representing cytoplasmic myofilaments. Myocytes are characteristically flattened out after 24 h in culture. FIG. 7B shows a patch of myocyte cytoplasm microscopically selected to study the [Ca²⁺] transients in these cells.

FIGS. 8A-8C illustrate [Ca²⁺] transients in intrinsic cardiac adrenergic cells. FIG. 8A shows the inhibition of [Ca²⁺] transients when extracellular Ca²⁺ is depleted. FIG. 8B shows the inhibition of [Ca²⁺] transients in the presence of tetrodotoxin (TTX), which specifically blocks voltage sensitive Na⁺ channels. FIG. 8C shows the decrease in amplitude of [Ca²⁺] transients in the presence of L-type calcium channel blocker nifedipine. Cells are excited at 340/380 nm.

FIGS. 9A-9D illustrate hypoxia/reoxygenation regulation of [Ca²⁺]_{I} transients in intrinsic cardiac adrenergic cells. FIGS. 9A and 9B show the inhibition and the subsequent rebound increase in activity after reoxygenation (Re—O₂) of [Ca²⁺]_{I} transients in intrinsic cardiac adrenergic cells when the cells are exposed to hypoxia for 3 and 10 minutes respectively. FIG. 9C shows elevated Ca²⁺ levels as a result of temporal summation of rapid [Ca²⁺] transient spiking following hypoxia (mean reduction in spike 2±1 to 0.2±0.1 spikes/min, P<0.001) and reoxygenation (mean increase in spike 2±1 to 13±4 spikes/min, P<0.001, n=10). FIG. 9D shows the high speed bursting of [Ca²⁺]i transients indicated in FIG. 9A (arrow).

FIGS. 10A-10E illustrate the presence of norepinephrine (NE) transporter (NET) in fetal rat intrinsic cardiac adrenergic cells co-cultured with myocytes. FIGS. 10A and 10B show the immunoreactivity of norepinephrine transporter and tyrosine hydroxylase respectively in the intrinsic cardiac adrenergic cells of fetal rat heart. The nuclei corresponding to intrinsic cardiac adrenergic cells and adjacent myocytes are shown in FIG. 10C. [³H]norepinephrine uptake and inhibition is shown in FIG. 10D. The uptake reaches 73±3 pg/mg protein per 2 h (n=6). This uptake is reduced by 20±5 and 36±4% in the presence of norepinephrine (1 μM) and nisoxetine (1 μM) respectively (n=6). FIG. 10E shows the percentage release of [³H] norepinephrine from intrinsic cardiac adrenergic cells at different time intervals. Scale bars=20 μm. *P<0.05 and **P<0.01.

FIGS. 11A-11C illustrate the adrenergic influence of intrinsic cardiac adrenergic cells on myocytes in intrinsic cardiac adrenergic cell-myocytes co-culture. FIG. 11A illustrates the dose-dependent reduction in the amplitude and frequency of [Ca²⁺]i transients generated by a myocyte in the presence of Atenolol (AT). At a concentration of 100 nM (red), Atenolol decreased myocyte beating rate from 66 to 7 beats/minute with a 40% reduction in the amplitude of [Ca²⁺]i transients. Atenolol when administered at 1 μM (green) stops the myocyte beating. FIGS. 11B and 11C illustrate the dose-dependent reduction in amplitude of [Ca^{2+]}i transients and beating rates of myocytes co-cultured with intrinsic cardiac adrenergic cells 9n=11) after administering different concentrations of Atenolol. *P<0.05 and **P.<0.01.

FIG. 12 illustrates the steps involved in the biosynthesis of catecholamines. DD, dopa decarboxylase; DBH, dopamine β-hydroxylase; Epi, epinephrine.

FIGS. 13A-13H show immunoperoxidase and immunofluorescent labeling of intrinsic cardiac adrenergic cells in human hearts. Intrinsic cardiac adrenergic cells expressing tyrosine hydroxylase immunoreactivity (red) are diffusely distributed in the LV myocardium (FIGS. 13A-13F) and the sinoatrial nodal tissue (FIG. 13G). Perivascular location is a frequent feature of intrinsic cardiac adrenergic cells. Arrows (FIGS. 13C and 13E) denote a terminal artery and vascular lumen, respectively. Tyrosine hydroxylase-expressing sympathetic nerve endings (arrows in FIGS. 13D and 13G) occasionally can be seen near intrinsic cardiac adrenergic cells. Inserts (FIGS. 13B and 13D) are the magnified images of intrinsic cardiac adrenergic cells in panels (FIG. 13B) (arrow) and (FIG. 13D), respectively. Note the low magnification (60×) image (FIG. 13B) compared to the rest of photomicrographs (100×). FIG. 13H shows an intrinsic cardiac adrenergic cell cluster in transplanted human ventricular tissue. All the scale bars are 10 μm except for FIG. 13B (20 μm).

FIGS. 14A-14F show in situ tyrosine hydroxylase mRNA expression in human intrinsic cardiac adrenergic cells. Two intrinsic cardiac adrenergic cells in FIG. 14A that exhibits tyrosine hydroxylase immunoreactivity (green) also express tyrosine hydroxylase mRNA (red, FIG. 14B) detected by in situ hybridization in the LV myocardium. Inserts are magnified images of intrinsic cardiac adrenergic cells. FIG. 14C shows tyrosine hydroxylase immunoreactivity (green) exhibited by a bundle of sympathetic nerve fibers expressing no tyrosine hydroxylase mRNA (FIG. 14D). FIGS. 14E and 14F show co-expression of protein gene product 9.5 (PGP 9.5) (green, FIG. 14E) and tyrosine hydroxylase (red, FIG. 14F) in a cluster of intrinsic cardiac adrenergic cells. Scale bars=10 mm.

FIGS. 15A-15H show δ-opioid receptor expression in human intrinsic cardiac adrenergic cells. Immunofluorescent co-localization of tyrosine hydroxylase (green, FIG. 15A) and δ-opioid receptor immunoreactivity (red, FIG. 15B) in an intrinsic cardiac adrenergic cell in human LV tissue. FIG. 15C: superimposed images of FIGS. 15A and 15B exhibiting tyrosine hydroxylase and δ-opioid receptor distributions with tyrosine hydroxylase concentrated on the opposite end. Inserts are magnified images of intrinsic cardiac adrenergic cells. FIGS. 15D and 15E show an intrinsic cardiac adrenergic cell co-expressing immunoreactivity of tyrosine hydroxylase and δ-opioid receptor. FIG. 15F shows an intrinsic cardiac adrenergic cell of perivascular distribution in LV tissue. FIG. 15G shows a tyrosine hydroxylase -expressing sympathetic nerve fiber that exhibits no δ-opioid receptor immunoreactivity on double labeling (FIG. 15H). Scale bars=10 μm.

FIGS. 16A-16H show δ-opioid receptor expression in rat and human intrinsic cardiac adrenergic cells. Immunofluorescent double labeling co-localizes δ-opioid receptor (red, FIG. 16A) and tyrosine hydroxylase (green, FIG. 16B) immunoreactivity in a cluster of intrinsic cardiac adrenergic cells in rat ventricular tissue. FIG. 16C shows dissociated rat cardiocytes with only intrinsic cardiac adrenergic cell but not myocytes exhibiting δ-opioid receptor immunoreactivity (green). The nuclei (blue color) of dissociated intrinsic cardiac adrenergic cell and myocytes are stained with DAPI. FIG. 16D displays δ-opioid receptor immunoreactivity (green) expressed by magnetically isolated intrinsic cardiac adrenergic cells (from dissociated cardiocytes) with >90% expressing δ-opioid receptor. Immunofluorescent double labeling (FIGS. 16E and 16F) co-localizes the δ-opioid receptor and TH immunoreactivity in isolated rat intrinsic cardiac adrenergic cells. FIG. 16G shows δ-opioid receptor activity (green) in two intrinsic cardiac adrenergic cells (arrow) abutted on muscle cells expressing muscle marker myosin heavy chain (MHC) (red) in human ventricular tissue section. Calibration bar=10 μm. FIG. 16H shows western blot analysis detects δ-opioid receptor protein in intrinsic cardiac adrenergic cell isolates (lane 1) and brain tissue (lane 2) but not in ventricular myocytes with depleted intrinsic cardiac adrenergic cells (lane 3). Protein loading was 25 μg/lane. The equivalent amount of protein loaded per lane was verified by β-actin levels.

FIGS. 17A-17B illustrates modulation of [Ca²⁺]i transients generated by intrinsic cardiac adrenergic cells by [D-Pen²⁵]-enkephalin (DPDPE). FIG. 17A: shows D-Penn²⁵]-enkephalin elicits a concentration-dependent increase in [Ca²⁺]i transients. Top tracing shows the excitatory effect of [D-Pen²⁵]-enkephalin on [Ca²⁺]i transients generated by an intrinsic cardiac adrenergic cell in culture. D-Pen²⁵]-enkephalin (100 nmol/L for 15 min) slowly increases the [Ca²⁺]i transients. The enhanced activity persists after the removal of D-Pen²⁵]-enkephalin. Bottom tracing shows another intrinsic cardiac adrenergic cell displaying initial inhibition of [Ca²⁺]i transients following by an excitatory phase after D-Pen²⁵]-enkephalin application (100 nmol/L for 15 min). Nifedipine reversibly abolishes DPDPE-mediated excitation in this intrinsic cardiac adrenergic cell. The average data in (FIG. 17B), shows that D-Pen²⁵]-enkephalin (100 nmol/L) increases [Ca²⁺]i transients in intrinsic cardiac adrenergic cells (n=5). In the presence of naltrindole (NTI, 1 mmol/L), D-Pen²⁵]-enkephalin (100 nmol/L) fails to increase [Ca²⁺]i transients (n=4). Tracing shows the lack of an excitatory effect of D-Pen²⁵]-enkephalin (100 nmol/L) on an intrinsic cardiac adrenergic cells pre-treated with Rp-CAMPS (100 μmol/L).

FIG. 18 shows that D-Pen²⁵]-enkephalin enhances epinephrine release from intrinsic cardiac adrenergic cells in culture. Basal epinephrine release at 1 hr is not different between the two groups before the treatment of D-Pen²⁵]-enkephalin or vehicle. Application of D-Pen²⁵]-enkephalin (+D-Pen²⁵]-enkephalin, 100 nmol/L for 30 min) to intrinsic cardiac adrenergic cells increases epinephrine release by 2.4 fold (n=4 duplicates). There is no increase in epinephrine release when intrinsic cardiac adrenergic cells are exposed to vehicle solution (D-Pen²⁵-enkephalin). Enhanced epinephrine release persists during recovery phase after the removal of D-Pen²⁵]-enkephalin.

FIGS. 19A-19C show the effect of D-Pen²⁵]-enkephalin on infarct size reduction in the presence and absence of labetalol. Bar graph shows standardized infarct size (infarct zone/area at risk of LV) for control (saline), D-Pen²⁵]-enkephalin, and labetalol +D-Pen²⁵]-enkephalin group (n=6/group). Saline and D-Pen²⁵]-enkephalin were injected 30-min before coronary artery occlusion. Labetalol was given 30-min before D-Pen²⁵]-enkephalin infusion. Photographs show typical samples of infarct zone in control FIG. 19A, D-Pen²⁵]-enkephalin-treated FIG. 19B and labetalol +-D-Pen²⁵]-enkephalin-treated FIG. 19C groups. The yellow and red colors denote the infarct zone and area at risk, respectively (**: p<0.01, *p<0.05).

FIG. 20 shows up-regulation of brain natriuretic peptide in cultured intrinsic cardiac adrenergic cells. Substantial up-regulation of brain natriuretic peptide (BNP) mRNA in cultured intrinsic cardiac adrenergic cells occurs following 1-hr treatment with d-opioid receptor agonist DPDPE, non-selective b-adrenergic receptor epinephrine, or selective b2-adrenergic receptor agonists terbutaline. Total RNA of intrinsic cardiac adrenergic cells was harvested 6-hr after the 1-hr drug treatment. BNP mRNA is quantified using real-time PCR.

### DETAILED DESCRIPTION OF THE INVENTION

The existence of δ-opioid receptors in the heart has been described for years. Receptor binding studies have demonstrated δ-opioid receptors in rat and human ventricular tissue. However, the exact cellular location of these receptors in the heart has not been identified. The present invention discloses the presence of δ-opioid receptors in the intrinsic cardiac adrenergic cells in fetal rat, adult rat, and adult human hearts. Immunostaining was used to show the presence of these receptors in intrinsic cardiac adrenergic cells. Stimulation of δ-opioid receptors by D-Pen²⁵]-enkephalin, a δ-opioid receptor agonist, was found to activate intrinsic cardiac adrenergic cells by enhancing Ca²⁺ influx through L-type calcium channels. This increase in Ca²⁺ influx is required for release of catecholamines such as adrenaline.

Endogenous catecholamines (mainly epinephrine) exert myocardial protection against myocardial ischemia and facilitate functional recovery after myocardial infarction. This effect is predominantly mediated through the activation of myocardial b₂-adrenoreceptors. The present invention demonstrates δ-opioid receptors are expressed exclusively by intrinsic cardiac adrenergic cells. Cardiac sympathetic nerve endings and cardiac myocytes do not express δ-opioid receptors. This suggests δ-opioid receptor agonists do not exert direct modulating effect on cardiac muscle cells. Stimulation of δ-opioid receptors of intrinsic cardiac adrenergic cells greatly enhances endogenous catecholamine release. Thus, myocardial protection associated with δ-opioid receptor stimulation is exclusively mediated by activation of intrinsic cardiac adrenergic cells with subsequently enhanced catecholamine release. The catecholamines derived from intrinsic cardiac adrenergic cells activate myocardial β2-adrenergic receptors resulting in myocardial protection against ischemic insult. This important discovery paves a way for developing novel therapeutic strategies for ischemic heart disease. Based on this discovery, strategies can specifically target δ-opioid receptors exclusively expressed by intrinsic cardiac adrenergic cells to mobilize endogenous catecholamine release right within the heart thereby postconditioning the myocardium during the ischemic insult.

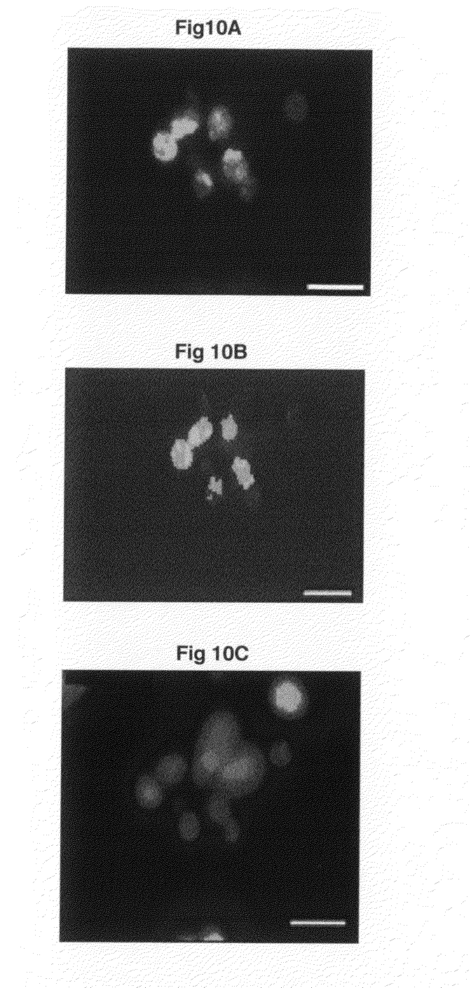

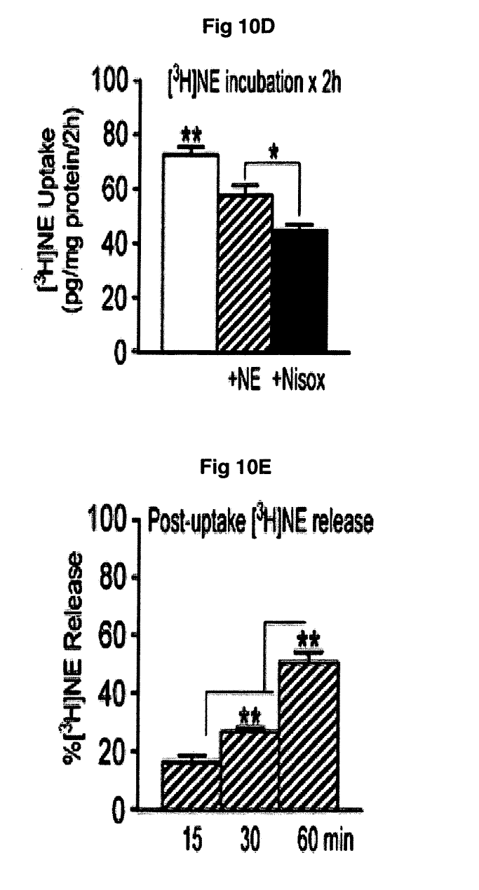

The invention in one embodiment discloses the effect of acute hypoxia and reoxygenation on intrinsic cardiac adrenergic cells. Acute hypoxia markedly inhibits [Ca²⁺]i transients of intrinsic cardiac adrenergic cells (FIGS. 10A-10C). This indicates hypoxia-mediated intrinsic cardiac adrenergic cell inactivation may have an important role in hypoxic bradycardia, presumably as a result of diminished catecholamine release. The histological evidence that intrinsic cardiac adrenergic cells are closely associated with cardiac pacemaking and conduction tissue further supports this contention. Reoxygenation (FIGS. 10A-10C) after hypoxia elicits an increase in [Ca²⁺]I transient frequency of intrinsic cardiac adrenergic cells. This may represent a highly effective mechanism for Ca²⁺ influx-dependent intrinsic cardiac adrenergic cells activation during reoxygenation.

The invention further discloses the stimulation of δ-opioid receptors of intrinsic cardiac adrenergic cells leads to an excessive release of adrenaline from these cells. Thus in one embodiment the invention provides a method for direct activation of intrinsic cardiac adrenergic cells by δ-opioid receptor specific drugs to enhance intra-cardiac adrenaline release for myocardial support of a patient suffering from acute decompensated congestive heart failure. Furthermore, in this embodiment the invention also provides a method for endogenous release of other protective neurohormones required to manage heart failure such as brain natriuretic peptide. Agonists of intrinsic cardiac adrenergic cell δ-opioid receptors such as [D-Pen²⁵]-enkephalin can be used to stimulate the release of endogenous catecholamines and brain natriuretic peptide in patients suffering from acute decompensated heart failure.

In one embodiment the present invention discloses activation of protein kinase A (PICA) and L-type Ca²⁺ channel is required for the opioid signaling pathway via the δ-opioid receptors of intrinsic cardiac adrenergic cells. The activation of membrane voltage sensitive Na⁺ channels is also necessary for generating [Ca²⁺]i transients by intrinsic cardiac adrenergic cells, as they fail to do so in the presence of tetrodotoxin, which specifically blocks voltage sensitive Na⁺ channels in excitable cells. Nifedepine, a L-type calcium channel blocker was found to reduce the amplitude of [Ca²⁺]i transients of intrinsic cardiac adrenergic cells. In the presence of Rp CAMP, a protein kinase A inhibitor, the excitatory action of [D-Pen²⁵-enkephalin is blocked. In this embodiment the present invention provides a method for treating heart failure in an individual using compounds that can inhibit or activate protein kinase A and/or increase or decrease [Ca²⁺]I transients in intrinsic cardiac adrenergic cells depending on the conditions characterizing the heart failure.

In one embodiment of the present invention, the up-regulation or stimulation of δ-opioid receptors of intrinsic cardiac adrenergic cells in the heart with subsequent enhanced endogenous cardiac catecholamine release may provide for a more effective and safer beta-adrenergic cardiac augmentation for the decompensated failing heart as compared to exogenous catecholamine treatment. This new approach in the treatment of congestive heart failure can minimize or eliminate the use of exogenous beta-adrenergic agonists like dopamine and dobutamine, which have been associated with increased mortality.

The present invention discloses a novel adrenergic signaling system involved in cardiac regulation. Immunohistochemical study of fetal rat hearts demonstrated the presence of intrinsic cardiac adrenergic cells with catecholamine biosynthetic enzymes, tyrosine hydroxylase and phenylethanolamine N-methyl transferase. The mRNA of tyrosine hydroxylase and phenylethanolamine N-methyl transferase was also detected in fetal rat hearts before sympathetic innervation using in situ hybridization techniques.

The findings of functional myocardial beta-receptors and catecholamine release from intrinsic cardiac adrenergic cells and its regulatory effect on [Ca²⁺]I transients of fetal myocytes provide compelling evidence of a highly effective intrinsic cardiac adrenergic cell signaling pathway that is critically important in early fetal development.

The invention also discloses the presence of norepinephrine transporter in intrinsic cardiac adrenergic cells of rat heart tissue. Nisoxetine, an norepinephrine transporter inhibitor only partially inhibited the uptake of norepinephrine by norepinephrine transporter present in intrinsic cardiac adrenergic cells. This suggests that norepinephrine transporter expressed in intrinsic cardiac adrenergic cells differ in structural and/or functional properties to the norepinephrine transporter expressed in sympathetic nerve endings.

Brain natriuretic peptide is a neuropeptide synthesized and released by the heart. Release of brain natriuretic peptide is greatly increased during disease states such as acute decompensated congestive heart failure. This brain natriuretic peptide release, is an important compensatory mechanism of the acute decompensated failing heart and intravenous infusion of brain natriuretic peptide has been used clinically to effectively treat acute severely decompensated heart failure. Although it was proposed that a special type of ventricular cell muscle releases brain natriuretic peptide, the exact cell type of brain natriuretic peptide releasing ventricular cell was not identified. The present invention discloses the synthesis of brain natriuretic peptide by intrinsic cardiac adrenergic cells. Immunohistochemical staining techniques were used to show the presence of brain natriuretic peptide in intrinsic cardiac adrenergic cells in rat and human hearts and not in other myocytes. Furthermore using in situ hybridization technique the presence of brain natriuretic peptide mRNA in intrinsic cardiac adrenergic cells was established. The presence of brain natriuretic peptide mRNA in intrinsic cardiac adrenergic cells eliminates the possibility the peptide was formed elsewhere and then internalized by these cells.

The production of brain natriuretic peptide by intrinsic cardiac adrenergic cells provides a mode for pharmacological manipulation of these cells to cause endogenous release of brain natriuretic peptide. Hypoxia-induced brain natriuretic peptide release in intact human and isolated rat hearts has been observed previously. Conceivably, there may be several potential mechanisms for manipulating circulating brain natriuretic peptide levels through the increased synthesis and release of brain natriuretic peptide or reduced brain natriuretic peptide degradation. Thus in one embodiment the invention discloses a method to stimulate excessive endogenous synthesis and release of brain natriuretic peptide from intrinsic cardiac adrenergic cells in an individual suffering from decompensated congestive heart failure. This innovative approach may achieve similar or better outcome in such patients as compared to exogenous infusion of the peptide both clinically and economically.

The increase in endogenous production of brain natriuretic peptide may also be beneficial in other diseases characterized by excessive fluid retention. Thus in yet another embodiment the invention presents a method of producing diuresis in an individual in need of such treatment by stimulating intrinsic cardiac adrenergic cells to release brain natriuretic peptide. An example of such a disease is cirrhosis.

Injection of morphine to patients with acute heart failure rapidly improves symptoms and clinical outcome. The mechanism underlying morphine-mediated symptomatic relief is not known. It is probable that morphine stimulates δ-opioid receptors in intrinsic cardiac adrenergic cells to increase brain natriuretic peptide production. The presence of δ-opioid receptors in intrinsic cardiac adrenergic cells provides a means of specifically stimulating these receptors to enhance brain natriuretic peptide production and exert cardioprotection in patients suffering from decompensated congestive heart failure. Accordingly, in one embodiment, the present invention provides a method for treating decompensated congestive heart failure in an individual by stimulating δ-opioid receptors for enhanced endogenous brain natriuretic peptide production.

The agents required for endogenous regulation of brain natriuretic peptide and catecholamines to treat heart failure can be administered via oral, intramuscular, intradermal or subcutaneous route. A pharmaceutical formulation of such agents may contain acceptable carriers and additives. These agents may also be administered with exogenous brain natriuretic peptide and/or catecholamines.

Thus, the present invention is directed to a method of treating an individual suffering from a cardiovascular disease, comprising the step of administering to said individual an effective dose of a compound that stimulates release of endogenous brain natriuretic peptide from intrinsic cardiac adrenergic cells. Representative cardiovascular diseases are decompensated congestive heart failure and myocardial ischemic disease. Preferably, the compound stimulates δ-opioid receptors in the cells to initiate synthesis and release of the peptide. The compound may be administered in any acceptable fashion including via an oral, an intramuscular, an intravenous, an intradermal, or a subcutaneous route. This method may further comprise the step of administering exogenous brain natriuretic peptide to the individual.

The present invention is further directed to a method of treating an individual suffering from a cardiovascular disease, comprising the step of administering to said individual a pharmacologically effective dose of a compound that up-regulates catecholamine synthesis and release from cardiac adrenergic cells. Generally, the compound stimulates δ-opioid receptors in the cells to up-regulate synthesis and release of the catecholamines such as norepinephrine and epinephrine. Representative examples of useful compounds in this method include but are not limited to [D-Pen²⁵]-enkephalin, a deltorphin, or D-Ala2-D-Leu5-enkephalin. This method may further comprise the step of administering exogenous synthetic catecholamines to the individual. Representative exogenous catecholamines include dobutamine, dopamine, norepinephrine, and epinephrine.

The present invention is also directed to a method of treating an individual suffering from a cardiovascular disease, comprising administering to the individual a pharmacologically effective dose of a compound that inhibits catecholamine synthesis and release from cardiac adrenergic cells where the heart of the individual is protected from excessive beta-adrenergic stimulation in chronic congestive heart failure. Generally, the compound stimulates δ-opioid receptors in the cells to down-regulate synthesis and release of the catecholamines. Representative examples of useful compounds include but are not limited to calcium channel blockers such as nifedipine compounds that inhibit activation of protein kinase A. Representative cardiovascular diseases are decompensated congestive heart failure and myocardial ischemic disease.

The present invention is directed to a method of treating an individual with a disease characterized by excessive fluid retention such as cirrhosis, comprising the step of administering to the individual a pharmacologically effective dose of a compound that stimulates release of brain natriuretic peptide to produce diuresis. In this method, the compound preferably stimulates δ-opioid receptors in the cells to initiate synthesis and release of the peptide. Representative examples of useful compounds include but are not limited to [D-Pen²⁵]-enkephalin, a deltorphin, or D-Ala2-D-Leu5-enkephalin.

The present invention is directed to a method of stimulating release of brain natriuretic peptide from intrinsic cardiac adrenergic cells in an individual in need of such treatment, comprising administering a pharmacologically effective dose of an δ opioid receptor agonist to the individual.

The present invention is directed to a method of inducing cardioprotection in an individual in need of such treatment, comprising administering a pharmacologically effective dose of a compound that activates intrinsic cardiac adrenergic cells. Generally, the compound induces epinephrine release from said cells, and is an agonist of δ-opioid receptors, increases Ca2+ transients in said cells, and activates protein kinase A, or a combination thereof. Generally, the cardioprotection is directed towards decompensated congestive heart failure or myocardial ischemic disease.

As used herein, the term, “a” or “an” may mean one or more. As used herein in the claim(s), when used in conjunction with the word “comprising”, the words “a” or “an” may mean one, or more than one. As used herein “another” or “other” may mean at least a second, or more of the same, or different claim element, or components thereof.

As used herein, the term “compound” or “agonist” or “antagonist” means a molecular entity of natural, semi-synthetic or synthetic origin that either activates or blocks, stops, inhibits, and/or suppresses the effects of drugs. The composition described herein can be administered independently, either systemically or locally, by any method standard in the art. Dosage formulations of the composition described herein may comprise conventional non-toxic, physiologically or pharmaceutically acceptable carriers, or vehicles suitable for the method of administration and are well known to an individual having ordinary skill in this art.

The composition described herein may be administered independently or in combination with an agonist or antagonist and may comprise one or more administrations to achieve, maintain, or improve upon a therapeutic effect. It is well within the skill of an artisan to determine dosage, or whether a suitable dosage of the composition comprises a single administered dose or multiple administered doses. An appropriate dosage depends on the subject's health, the treatment, or prevention of effects of the stimulant drug, the route of administration, and the formulation used.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Preparation of Cardiac Cell Culture

Myocyte-intrinsic cardiac adrenergic cell cultures (E16) were prepared. The dissociated cardiocytes were pre-plated in medium containing bovine serum albumin, which allows fibroblasts and endothelial cells to attach to the plate. The cell suspension of the subsequent adherent culture is poured off to remove fibroblasts and endothelial cells, enriching for a primary population of myocytes and intrinsic cardiac adrenergic cells in the subsequent culture.

EXAMPLE 2

### Immunohistochemical Study

Immunofluorescent staining is performed on 3 mm paraffin sections of 4% paraformaldehyde fixed cardiac tissue. For double staining of tyrosine hydroxylase-brain natriuretic peptide and tyrosine hydroxylase-δ-opioid receptors in intrinsic cardiac adrenergic cells, tissue sections are incubated with anti-brain natriuretic peptide and tyrosine hydroxylase-δ-opioid receptor antibody (1:500, Chemicon) for 1 hr at 25° C. After washing, the slide is incubated with a second antibody for 1 hr. The double stain is completed by incubating the slide with anti-tyrosine hydroxylase antibody (1:50) overnight at 4° C. followed by incubation with the secondary antibody.

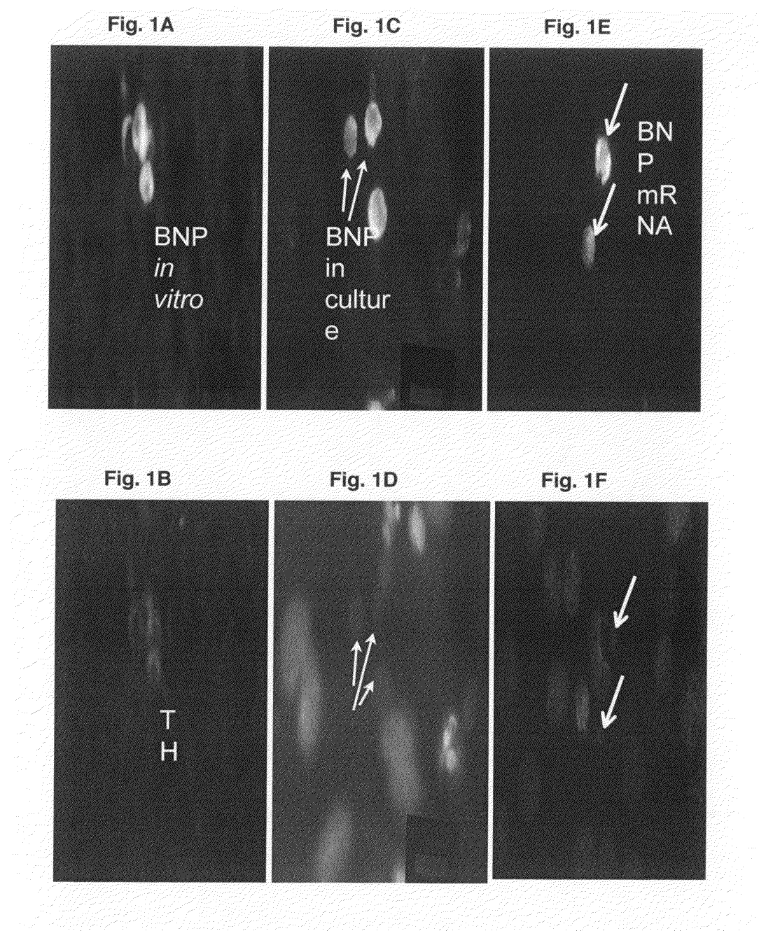

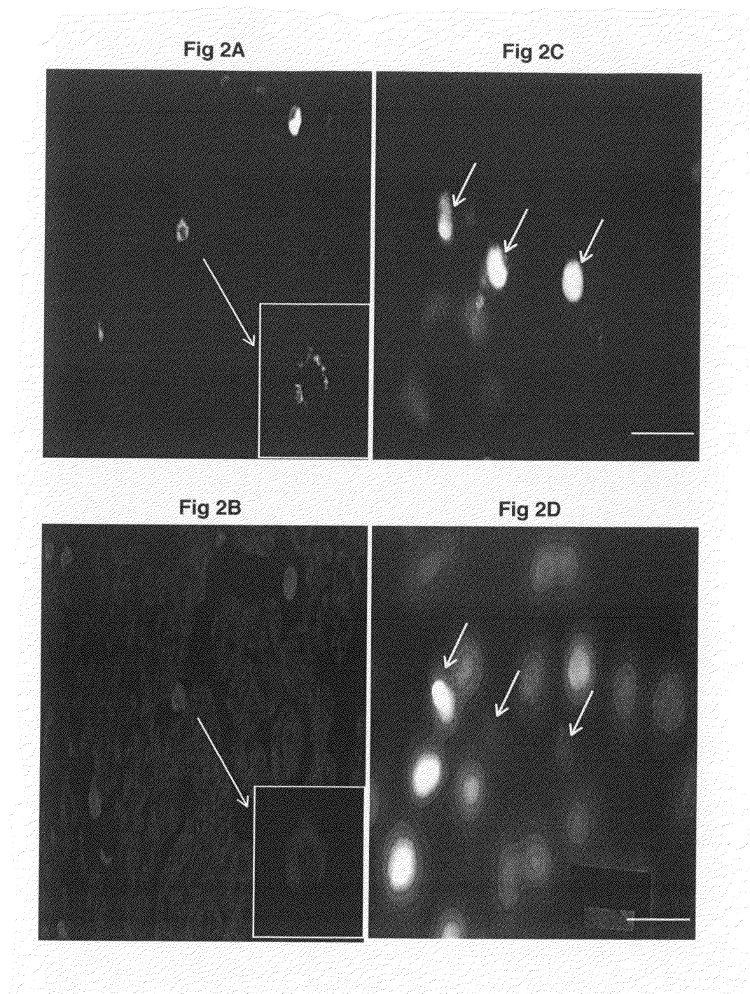

Immunohistochemical study showed that intrinsic cardiac adrenergic cells co-express brain natriuretic peptide and tyrosine hydroxylase in rat heart (FIGS. 1A-1D). Tyrosine hydroxylase, is a specific cell marker for adrenergic endocrine cells. The mRNA of brain natriuretic peptide was localized to intrinsic cardiac adrenergic cells but not to myocytes (FIGS. 1E and 1F). The evidence of brain natriuretic peptide protein and its mRNA in intrinsic cardiac adrenergic cells provides a definitive evidence of brain natriuretic peptide production site in the heart. Immunoreactivity of δ-opioid receptors was also identified in intrinsic cardiac adrenergic cells expressing tyrosine hydroxylase in rat ventricular tissue and in cardiac cell cultures (FIGS. 2A-2D).

### EXAMPLE 3

### Adrenergic Gene Expression in Fetal Heart

The mRNA from fetal rat hearts at embryonic day 16 (E16) and from maternal adrenal glands is isolated using TRIZOL®. Total RNA is reverse transcribed into cDNA using the first-strand synthesis kit (Invitrogen). The cDNA is reverse transcribed with primers 5′ AACTCTCCACGGTGTACTGGTT 3′ (forward) and 5′ GCATAGTTCCTGAGCTTGTCCT 3′ (reverse) for tyrosine hydroxylase (TH) and 5′ ACTGGAGTGTGTATAGCCAGCA 3′ (forward) and 5′ ACACTGGAACCACAGATAGCCT 3′ (reverse) for phenylethanolamine N-methyl transferase.

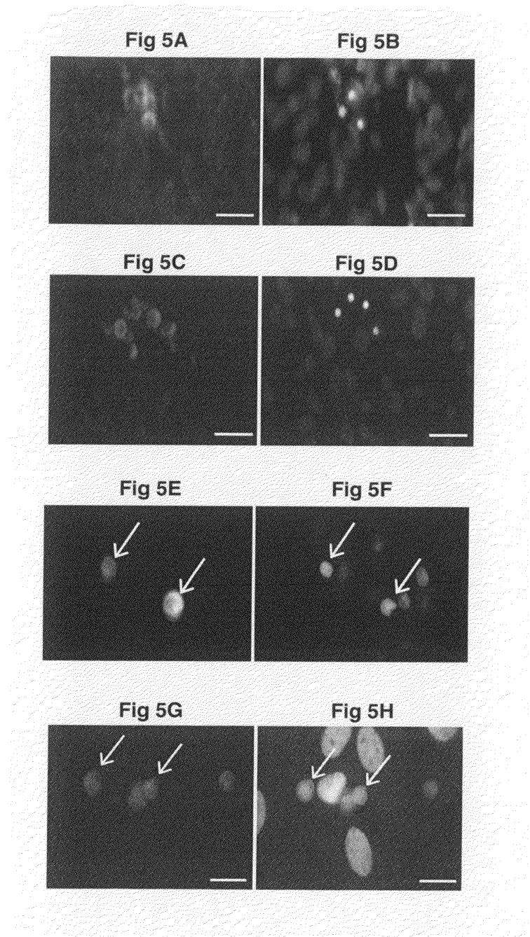

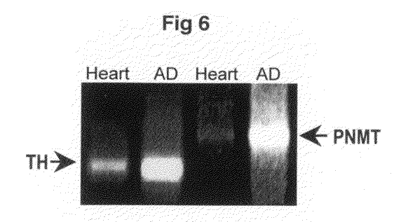

The expression of mRNA of tyrosine hydroxylase and phenylethanolamine N-methyl transferase was detected in fetal heart at E16 when no sympathetic innervation was detected (FIGS. 5A and 5C). FIGS. 5E and 5G show the immunoreactivity of tyrosine hydroxylase and phenylethanolamine N-methyl transferase in intrinsic cardiac adrenergic cell-myocyte co-cultures respectively. The PCR products of fetal heart mRNA for tyrosine hydroxylase and phenylethanolamine N-methyl transferase matched the maternal adrenal gland products included as a positive control (FIG. 6).

### EXAMPLE 4

### [Ca²⁺]i Transients in Intrinsic Cardiac Adrenergic Cells

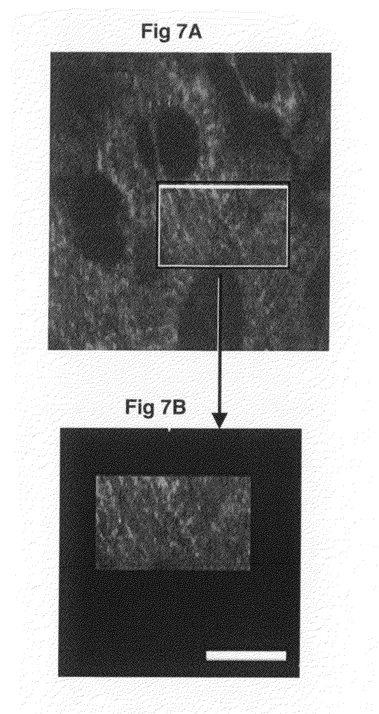

The intrinsic cardiac adrenergic cells in intrinsic cardiac adrenergic cell-myocyte co-culture (FIGS. 7A-7B) preparations generated spontaneous [Ca²⁺]i transients with markedly irregular rhythm. The spike frequency of [Ca²⁺]i transients recorded from a total of 42 cells varied with a mean rate of 5±4 spikes/min. The morphology of [Ca²⁺]I transients was characterized by a rapid upstroke with varied down sloping phase (cystolic calcium removal). The [Ca²⁺]I transients of intrinsic cardiac adrenergic cells were abolished after administration of calcium free solution (5±2 to 0 spike/min, n=5), or tetrodotoxin at 10 mM concentration (11±7 to 0 spikes/min, n=6). Nifedipine in the concentration range of 1 and 10 mM, reduced the amplitude of [Ca²⁺]i transients of intrinsic cardiac adrenergic cells by 54±8 and 82±3% (p<0.01, n=8), respectively. Atenolol at 1000 nM did not significantly increase the frequency of [Ca²⁺]i transients of intrinsic cardiac adrenergic cells. w-Conotoxin and w-Agatoxin IVA (both 30 mM) did not affect the [Ca²⁺]i transients of intrinsic cardiac adrenergic cells.

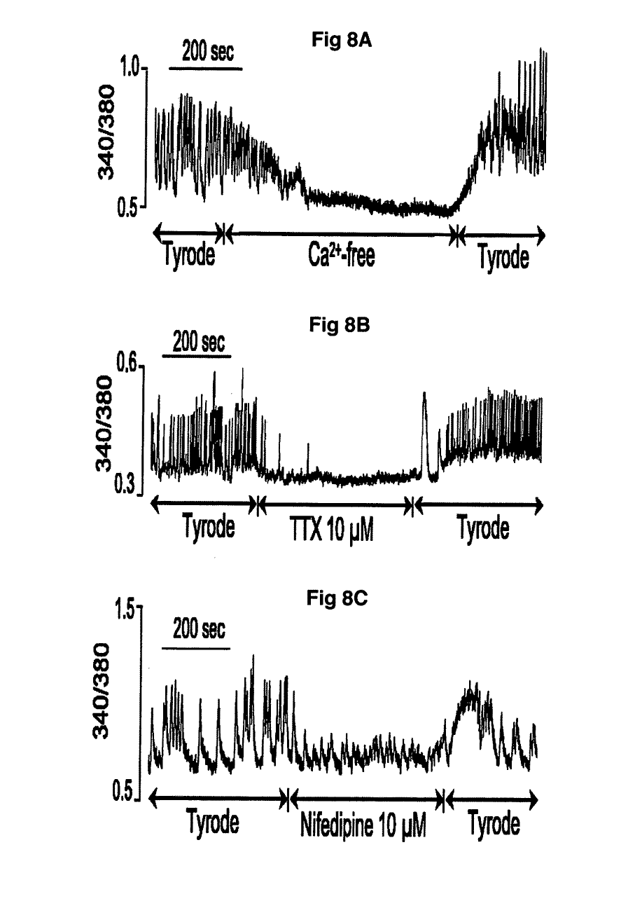

Calcium influx is the fundamental mechanism required for neurotransmitter release from neuroendocrine cells. The calcium influx-mediated [Ca²⁺]i transients of intrinsic cardiac adrenergic cells provide a physiological basis required for catecholamine release. The activation of membrane voltage sensitive Na⁺ channels is necessary for generating intrinsic cardiac adrenergic cell [Ca²⁺]i transients, since they fail to do so in the presence of tetradotoxin, which specifically blocks voltage sensitive Na channels in excitable cells (FIG. 8B). Nifedipine, a L-type calcium channel blocker reduces the amplitude of [Ca²⁺]i transients of intrinsic cardiac adrenergic cells (FIG. 8C). The role of N and P type calcium channels in the genesis of [Ca²⁺]i transients of intrinsic cardiac adrenergic cells was determined by administration of the respective blockers, w-Conotoxin and w-agatoxin IVA to the cells. These compounds did not affect [Ca²⁺]i transients in intrinsic cardiac adrenergic cells indicating that these calcium channels are not active in generating intrinsic cardiac adrenergic cell [Ca²⁺]i transients.

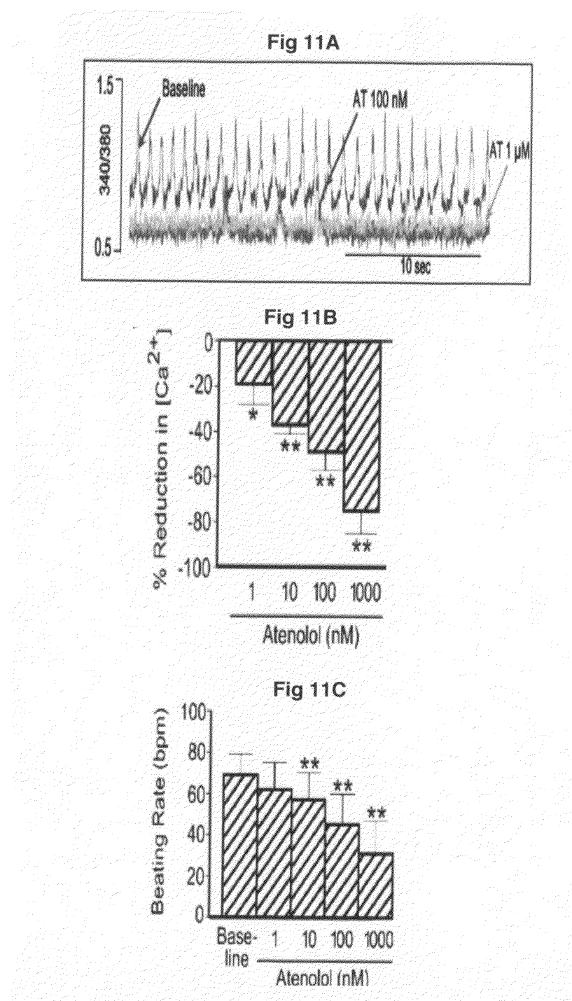

The unique pattern of [Ca²⁺]i transients generated by intrinsic cardiac adrenergic cells rules out the possibility such [Ca²⁺]i transients were recorded from contaminated myocytes in the co-cultures. This argument is supported by the evidence that basal frequency of [Ca²⁺]i transients of intrinsic cardiac adrenergic cells is 10 times slower than that of myocytes. Furthermore, the rhythm of [Ca²⁺]i transients of intrinsic cardiac adrenergic cells is irregular as compared to the regular beating rhythm generated by myocytes. Myocytes also do not produce bursting activity in [Ca²⁺]i transients as observed in intrinsic cardiac adrenergic cells. In intrinsic cardiac adrenergic cells, [Ca²⁺]i transients are not significantly affected in the presence of Atenolol, which greatly reduced the beating frequency of myocytes (FIGS. 11A-11C).

Intrinsic cardiac adrenergic cells isolated from myocytes using a magnetic bead purification method displayed the same immunohistological characteristics and generated a similar pattern of spontaneous [Ca²⁺]i transients as those intrinsic cardiac adrenergic cells co-cultured with myocytes.

### EXAMPLE 5

### Effects of Acute Hypoxia and Reoxygenation of Intrinsic Cardiac Adrenergic Cells

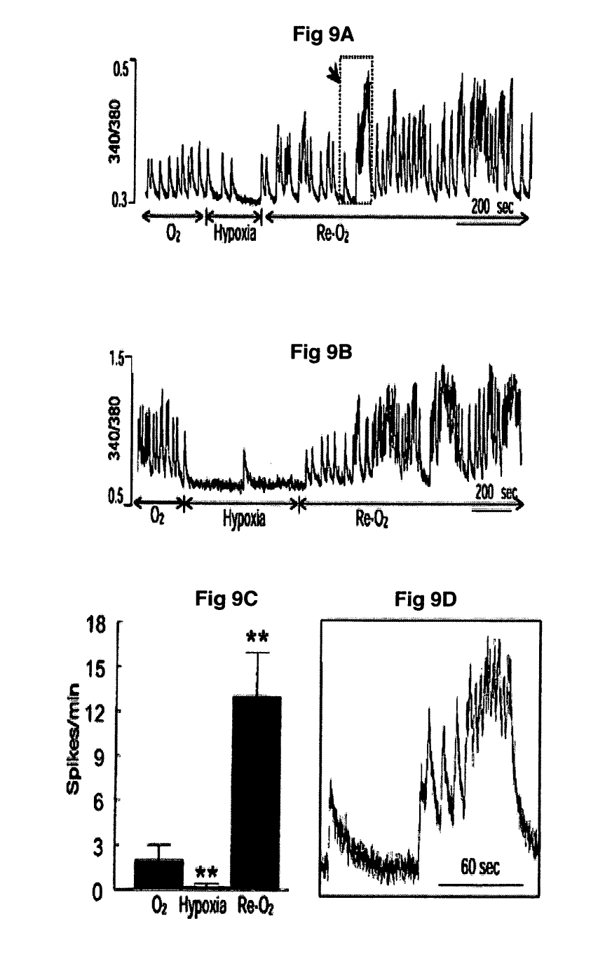

Acute hypoxia inhibited [Ca²⁺]i transients with a rapid onset. Inhibition of intrinsic cardiac adrenergic cells may act in coordination with autonomic reflex mechanisms to reduce myocardial oxygen consumption during acute hypoxia through the reduction of cardiac catecholamine release. The inhibitory response of intrinsic cardiac adrenergic cells to hypoxia (FIG. 9A), distinguishes them from adrenal chromaffin cells, which exhibit an excitatory response to hypoxia resulting in enhanced catecholamine release. Such different responses to hypoxia may represent tissue-specific differences between sympathoadrenal neurons and intrinsic cardiac adrenergic cells.

Re-oxygenation of hypoxic intrinsic cardiac adrenergic cells immediately exerted a potent stimulatory effect on intrinsic cardiac adrenergic cells with a maximum six-fold increase in [Ca²⁺]i transient frequency (FIG. 9B). The stimulatory effect was rapid and sustained up to 20 minutes. Burst activity producing temporal summation of [Ca²⁺]i transients represents a distinct intrinsic cardiac adrenergic response to re-oxygenation (FIGS. 9C-9D). Burst response occurs when [Ca²⁺]i transient spikes arrive in quick succession, so that each adds to the preceding one, producing sustained calcium influx.

### EXAMPLE 6

### δ-Opioid Receptor Signaling

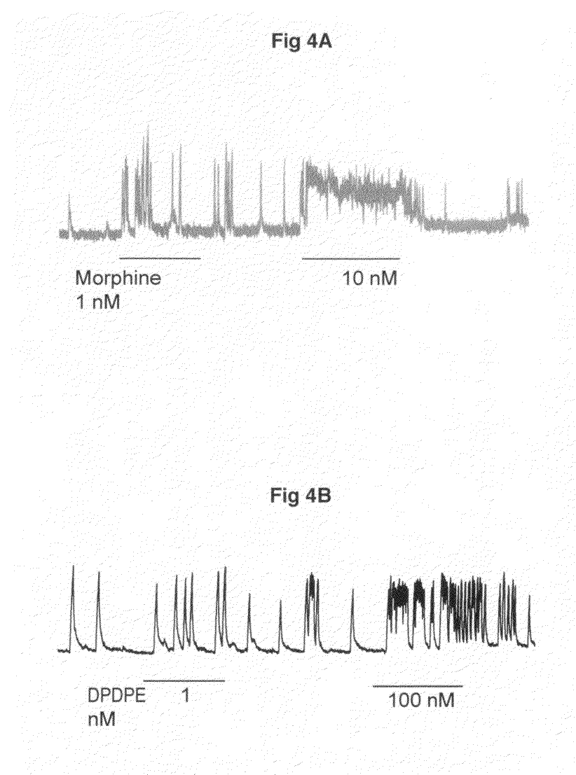

To determine δ-opioid receptor signaling mechanism the receptor agonist, [D-Pen²⁵]-enkephalin at concentrations ranging from 1-1000 nM was used to stimulate intrinsic cardiac adrenergic cells in the absence and presence of δ-opioid receptor antagonist NPI (1 nM). Intrinsic cardiac adrenergic cells failed to generate enhanced [Ca²⁺]i transients in the presence of the receptor antagonist. In the absence of the antagonist, [D-Pen²⁵ ]-enkephalin, elicited a 5-fold increase in [Ca²⁺]i transients generated by the intrinsic cardiac adrenergic cells (FIG. 4B). These results indicate that δ-opioid receptors are present in intrinsic cardiac adrenergic cells and responsible for generating [Ca²⁺]i transients. Enhancement of [Ca²⁺]i transients generated by intrinsic cardiac adrenergic cells was also seen in the presence of morphine, which is a non-specific opioid receptor agonist (FIG. 4A).

### EXAMPLE 7

### Role of Protein Kinase A (PKA) in δ-Opioid Receptor Signaling

Intrinsic cardiac adrenergic cells were treated with a protein kinase A inhibitor, RP-cAMP (10 mM). In the presence of protein kinase A inhibition, the stimulatory effect of [D-Pen²⁵]-enkephalin was re-examined. [D-Pen²⁵]-enkephalin failed to activate intrinsic cardiac adrenergic cells in the presence of protein kinase A inhibition. This indicates protein kinase A activation is required for the δ-opioid receptor mediated activation of intrinsic cardiac adrenergic cells.

### EXAMPLE 8

### Role of L-Type Calcium Channels in δ-Opioid Receptor Signaling

Activation of protein kinase A leads to the phosphorylation of L-type calcium channels. This indicates the L-type calcium channels may be involved in δ-opioid receptor signaling. To determine the role of these calcium channels in δ-opioid receptor signaling, the stimulatory effect of [D-Pen²⁵]-enkephalin on [Ca²⁺]i transients of intrinsic cardiac adrenergic cells is examined in the presence of L-type calcium channel blocker, Nifedipine (10 nM) (FIG. 8C). The stimulatory effect of [D-Pen²⁵]-enkephalin to increase [Ca²⁺]i transients was lost in the presence of Nifedipine, proving that L-type calcium channels have a primary role in the generation of ]Ca²⁺]i transients in intrinsic cardiac adrenergic cells.

### EXAMPLE 9

### [³H] Norepinephrine Uptake and Release Assay

[³H] norepinephrine uptake and release assays were performed in intrinsic cardiac adrenergic cell-monocyte co-cultures on a 24-well plate with a cell density of 2.5×10⁵/well after 24 hours in culture. The cells were incubated with 50 nmol/l [³H] norepinephrine (50 Ci/mmol, Amersham International) supplemented with ascorbic acid at 0.2 mmol/l in Tyrode solution for 2 hours. Excess [³H] norepinephrine was removed with six washes. To determine whether [³H] norepinephrine uptake can be inhibited by norepinephrine and a norepinephrine transporter inhibitor, the uptake studies were performed in the presence of norepinephrine (1 mM) and the norepinephrine transporter inhibitor nisoxetine (1 mM, Sigma) for 2 hours, respectively. [³H] norepinephrine uptake was defined as the sum of released and unreleased radioactivity. The magnitude of [³H] norepinephrine release was expressed as percent release from its total uptake.

Intrinsic cardiac adrenergic cells expressed immunoreactivity for the norepinephrine transporter (FIGS. 10A-10C). Norepinephrine transporter-mediated norepinephrine uptake was demonstrated by exogenous norepinephrine that competitively inhibited [³H] norepinephrine uptake by 20% (FIG. 10D). Nisoxetine only partially blocked [³H] norepinephrine uptake by intrinsic cardiac adrenergic cells suggesting the norepinephrine transporter expressed by intrinsic cardiac adrenergic cells may not have the same structural and/or functional properties as that located in sympathetic nerve endings (FIG. 10D). Thus this example demonstrates a novel adrenergic neuroendocrine system that possesses an active norepinephrine uptake mechanism in the heart independent of sympathetic innervation.

### EXAMPLE 10

### Importance of Intrinsic Cardiac Adrenergic Cells in Maintaining Cardiac Adrenergic Supply

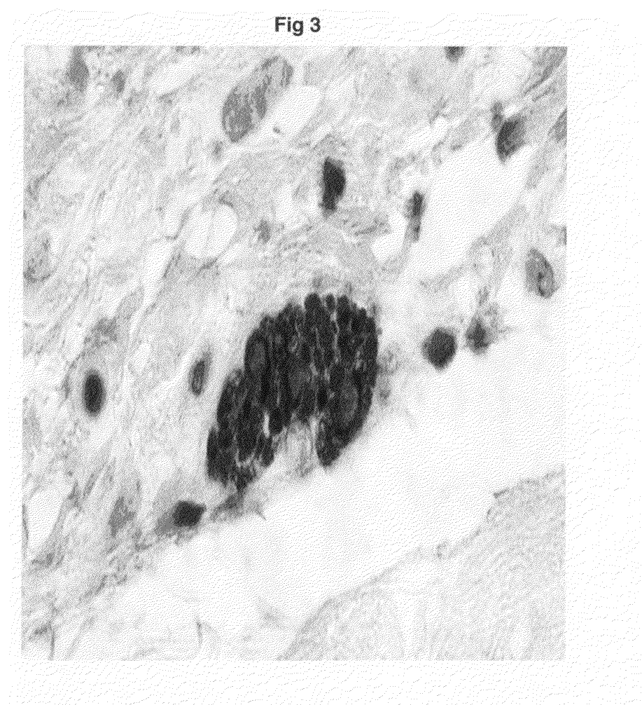

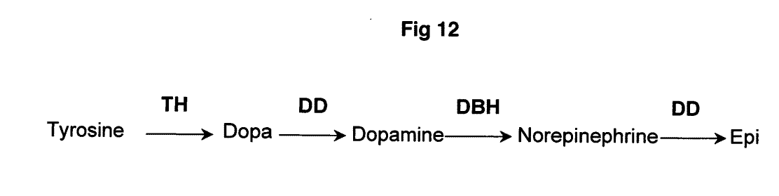

Adrenergic gene expression is obligatory for fetal survival before cardiac sympathetic innervation. The detection of mRNA of tyrosine hydroxylase and phenylethanolamine N-methyl transferase in the heart and localization of their respective enzyme proteins in intrinsic cardiac adrenergic cells in the absence of tyrosine hydroxylase-positive nerve endings demonstrate intrinsic cardiac adrenergic cells possess a well developed catecholamine synthetic system in the heart before sympathetic innervation (FIG. 3). These findings suggest intrinsic cardiac adrenergic cells provide an obligatory adrenergic supply to maintain cardiac function in early fetal development. Furthermore, the presence of intrinsic cardiac adrenergic cells in adult rat and human hearts support the concept that mammalian hearts possess an intrinsic cardiac adrenergic cell system throughout adult life. The findings of functional myocardial beta-receptors and norepinephrine release from intrinsic cardiac adrenergic cells and its regulatory effect on [Ca²⁺]i transients of fetal myocytes provide compelling evidence of a highly effective intrinsic cardiac adrenergic cell signaling pathway that is critically important in early development. The steps involved in the biosynthesis of catecholamines are illustrated in FIG. 12.

### EXAMPLE 11

Effect of b₁-Adrenergic Blockade on Myocyte [Ca²⁺]I Transients

At baseline, myocytes co-cultured (FIGS. 8A-8B) with intrinsic cardiac adrenergic cells generate rhythmic beat-to-beat [Ca²⁺]i transients (60±8 spikes/min). These fast and rhythmic [Ca²⁺]i transients generated by myocytes are distinctly different from those of intrinsic cardiac adrenergic cells, with a [Ca²⁺]i spike frequency of only 5±4 spikes/min. in the same culture. The influence of catecholamines derived from intrinsic cardiac adrenergic cells on [Ca²⁺]i transients of monocytes was assessed by the administration of atenolol to intrinsic cardiac adrenergic cell-myocyte co-cultures (FIGS. 11A-11C). Atenolol at 1,10,100 and 1000 nM reduced the amplitude of myocyte [Ca²⁺]i transients by 19±9, 37±4 , 49±8, and 75±10, with a concurrent decrease in beating rate by 16±7, 26±8, 57±11, and 62±13, respectively.

### EXAMPLE 11

### In Situ Hybridization for Brain Natriuretic Peptide Gene Expression in Human Intrinsic Cardiac Adrenergic Cells

RNA from human heart tissue is isolated using TRIZOL®. Total RNA is reverse transcribed into cDNA using the first-strand synthesis kit (Invitrogen). Primers for brain natriuretic peptide are designed using the sequence of the human brain natriuretic peptide gene (GENBANK accession number NM_{—}031545).

Forward primer: 5′ TGACGGGCTGAGGTTGTTTTAG 3′
Reverse primer: 5′ GGCAAGTTTGTGCTGGAAGATAAG 3′

The amplified brain natriuretic peptide fragment is resolved on a 2% agarose gel and the band corresponding to brain natriuretic peptide is excised and subcloned in frame in the pCRII vector (Invitrogen) containing a T3 RNA polymerase site. The RNA probe is generated using T3 RNA polymerase (Roche) and is ethanol precipitated. Heart tissue sections are prepared as 3 mm frozen sections. Prior to hybridization, tissue section is fixed, dehydrated and rehydrated. In situ hybridization is performed as previously reported.

### EXAMPLE 12

### Measurement of [Ca²⁺]I Transients of Intrinsic Cardiac Adrenergic Cells

Cultured rat intrinsic cardiac adrenergic cells are loaded with 4 mM fura 2-AM. Intrinsic cardiac adrenergic cells are washed three times and the cover slip is mounted on a temperature controlled (37° C.) chamber (Bioptechs) on the stage of an inverted microscope. A ratio-based fluorescent spectrophotometer is used for studying [Ca²⁺]I transients. A single isolated intrinsic cardiac adrenergic cell is excited at 340/380 nm alternatively at 15 Hz sampling rate. Measurements of 510 nm emissions from cells are recorded as ratios (340/380 nm) that are used to represent cystolic Ca²⁺ charges.

### EXAMPLE 13

### Radioimmunoassays for Brain Natriuretic Peptide

Radioimmunoassay for brain natriuretic peptide is performed according to the general protocol for Radioimmunoassay kit (Peninsula Laboratory). Isolated intrinsic cardiac adrenergic cells (2×10⁴/plate) in Tyrode (400 xx) are treated with [D-Pen²⁵]-enkephalin (100 nM) for either 10,30, or 60 minutes. Brain natriuretic peptide is measured using 100 μl of standard brain natriuretic peptide and 100 μl of intrinsic cardiac adrenergic cell-conditioned medium are pre-incubated with 100 μl of anti-serum for 24 hr at 4° C. 100 μl of ¹²⁵I-brain natriuretic peptide solution is added and incubated for 24 hr at 4° C. 100 μl of goat anti-rabbit IgG serum and 100 μl of normal rabbit serum are added and incubated at 37° C. for 1 hour followed by addition of radioimmunoassay buffer. The radioactivity in the samples is determined with a gamma counter.

### EXAMPLE 14

### Identification of Human Intrinsic Cardiac Adrenergic Cells

Immunohistochemical labeling of intrinsic cardiac adrenergic cells was performed on 4-μm paraffin sections of buffered 4% formaldehyde-fixed cardiac tissue, as described previously (Huang et al, 1996; Huang et al, 2005). Human heart tissue (n=5) was obtained from recipient's hearts during heart transplant surgery or autopsy. Four adult and four fetal rat (embryonic day 14) hearts were also studied. The protocols for using human and animal tissue were approved by the Institutional Review Board and the Institutional Animal Care and the Use Committee of the University of Texas Medical Branch. Tissues were taken from the LV free wall, septum, sinoatrial and atrioventricular nodal regions. Immunoperoxidase and immunofluorescent labeling were performed with an antibody against tyrosine hydroxylase (TH), a marker of the intrinsic cardiac adrenergic cells (Huang et al, 1996; Fryer et a1,1998; Fryer et al, 2001). The dilutions for mouse anti-human tyrosine hydroxylase (Neuromics, Northfield, Minn.) and mouse anti-rat tyrosine hydroxylase were 1:40. To co-localize δ-opioid receptor and tyrosine hydroxylase in intrinsic cardiac adrenergic cells, immunofluorescent double-labeling methods were used. The concentrations for rabbit anti-human δ-opioid receptor (US Biological, Swampscott, Mass.) and rabbit anti-rat δ-opioid receptor (Oncogene, San Diego, Calif.) were 1:200 and 1:250, respectively. The specificity of mouse anti-tyrosine hydroxylase and rabbit anti-δ-opioid receptor antibodies was tested by substituting these antibodies with Universal Negative Controls for Mouse and Rabbit IgG (DAKO Corporation, Carinteria, Calif.), respectively. Additionally, immunofluoresent double-labeling methods were used to determine whether human intrinsic cardiac adrenergic cells express neuronal marker PGP 9.5 or muscle marker myosin heavy chain (MHC). The dilutions for PGP 9.5 and myosin heavy chain were 1:3000 and 1:250, respectively. The double-staining study included four steps: (1) rabbit anti-δ-opioid receptor served as the first primary antibody and was stained with goat anti-rabbit ALEXA FLUOR® 594 followed by amplification with donkey anti-goat ALEXA FLUOR® 594; (2) slides were then incubated with biotin-labeled goat anti-rabbit for 30 min to saturate unbound rabbit IgG; (3) mouse anti-tyrosine hydroxylase served as the second primary antibody and was stained sequentially with rabbit anti-mouse ALEXA FLUOR® 488 and goat anti-rabbit ALEXA FLUOR® 488 (Signal-Amplification Kit for Mouse antibodies, Molecular Probes, Inc., Eugene, Oreg.). A control slide with omitted mouse anti-tyrosine hydroxylase treatment was stained with Streptavidin-ALEXA FLUOR® 488 after step (2) to test possible cross-reaction between goat anti-rabbit ALEXA FLUOR® 488 used in step (3) and rabbit anti-δ-opioid receptor antibody used in step (1); and (4) slides were counter-stained with DAPI. Double labeling of tyrosine hydroxylase with PGP 9.5 was performed in the same fashion. IMAGE-IT™ FX Signal Enhancer (Molecular probes, Inc., Eugene, Oreg.) and auto-fluorescence eliminator reagent were used before and after staining to block non-specific staining from background and auto-fluorescence. Co-localization of tyrosine hydroxylase and δ-opioid receptor was performed in rat hearts and in isolated rat intrinsic cardiac adrenergic cells in culture.

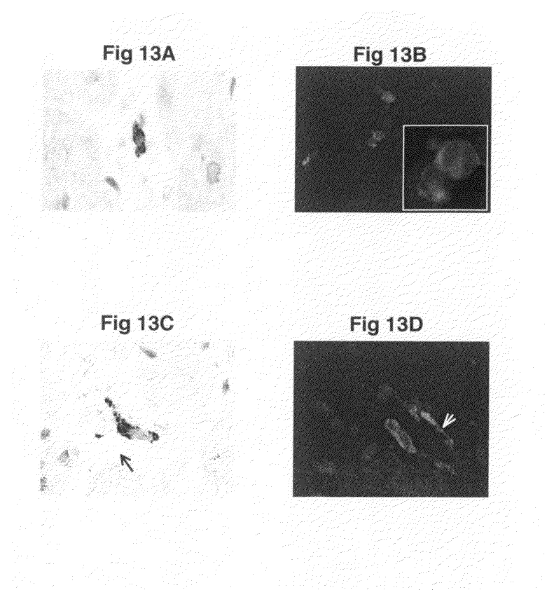

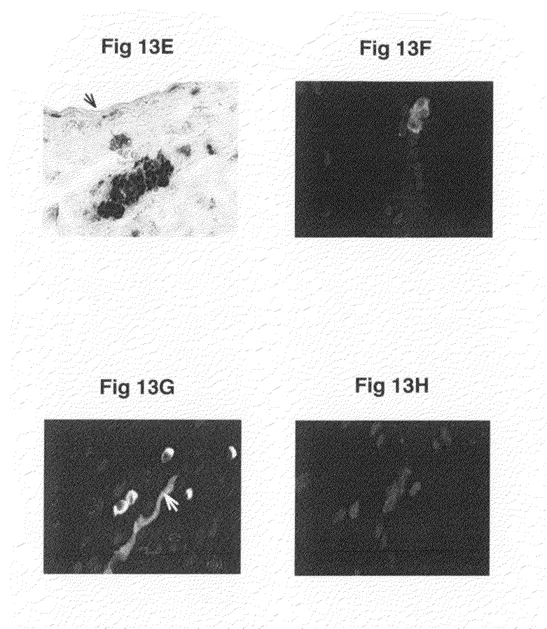

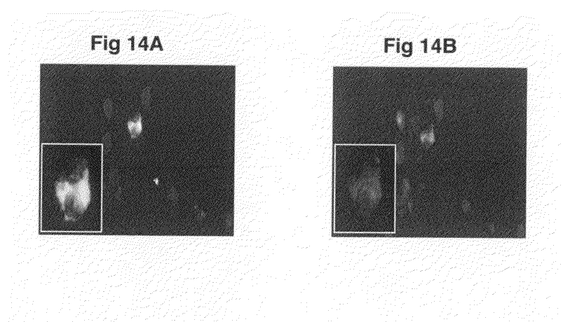

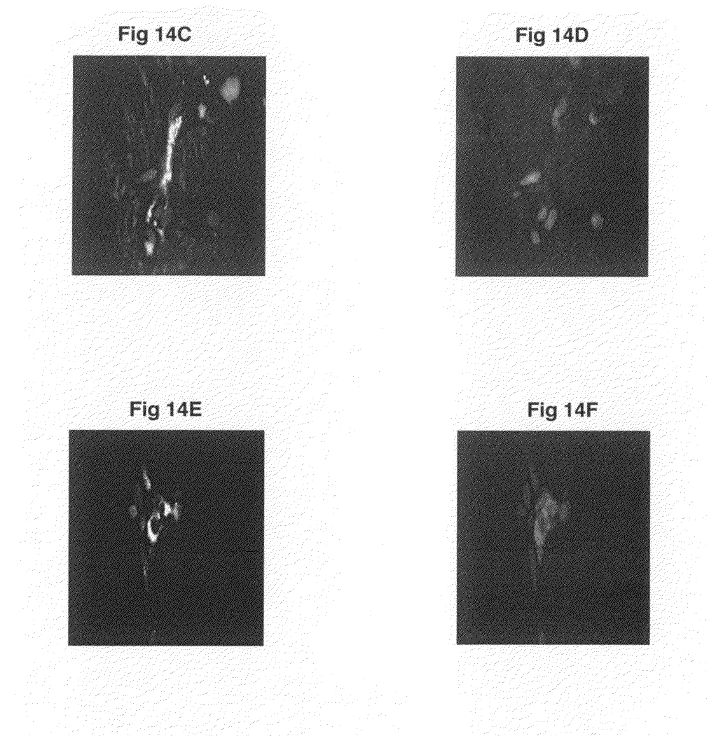

Intrinsic cardiac adrenergic cells were identified in the human LV myocardium (FIG. 13). In situ expression of tyrosine hydroxylase mRNA was identified in intrinsic cardiac adrenergic cells, but not in the sympathetic nerve endings of human hearts (FIG. 14). Intrinsic cardiac adrenergic cells are small (7-10 μm) with large nuclei and express tyrosine hydroxylase and neuronal marker PGP 9.5 (FIG. 14E). Frequently they form small clusters spreading in myocardial interstitium, and perivascular distribution is a common feature. The density of intrinsic cardiac adrenergic cells varied, constituting as many as 14% to as little as 0% of total cardiac cells per high magnified microscopic view. Intrinsic cardiac adrenergic cells were identified in human sinoatrial (FIG. 13G) and atrioventricular nodal tissues. Intrinsic cardiac adrenergic cells and sympathetic nerve endings were occasionally observed in the same loci (FIGS. 13D and 13G). Intrinsic cardiac adrenergic cells were identified in LV myocardium of transplanted hearts (FIG. 13H).

### EXAMPLE 15

Identification of d-Opioid Receptor in Intrinsic Cardiac Adrenergic Cells

To determine whether both intrinsic cardiac adrenergic cells and ventricular myocytes express d-opioid receptor, freshly isolated intrinsic cardiac adrenergic cells and myocytes with depleted intrinsic cardiac adrenergic cells were lysed separately to extract their protein. Protein of rat brain tissue served as positive control. Western blot analysis was performed as described (Huang et al, 1996). The dilution for rabbit anti-d-opioid receptor was 1:500.

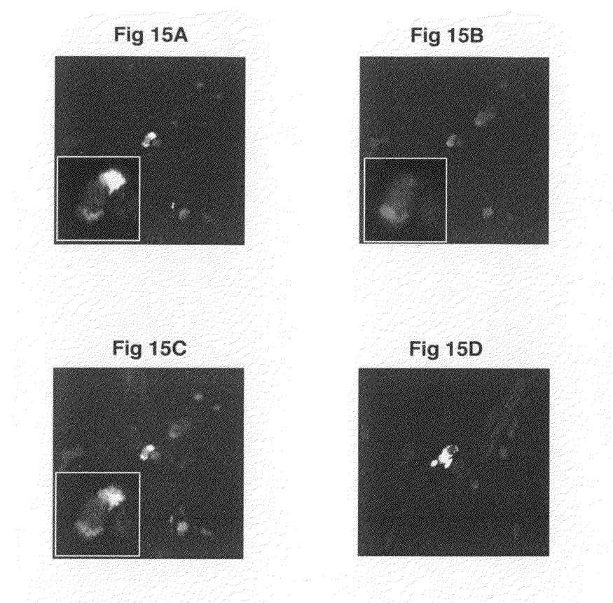

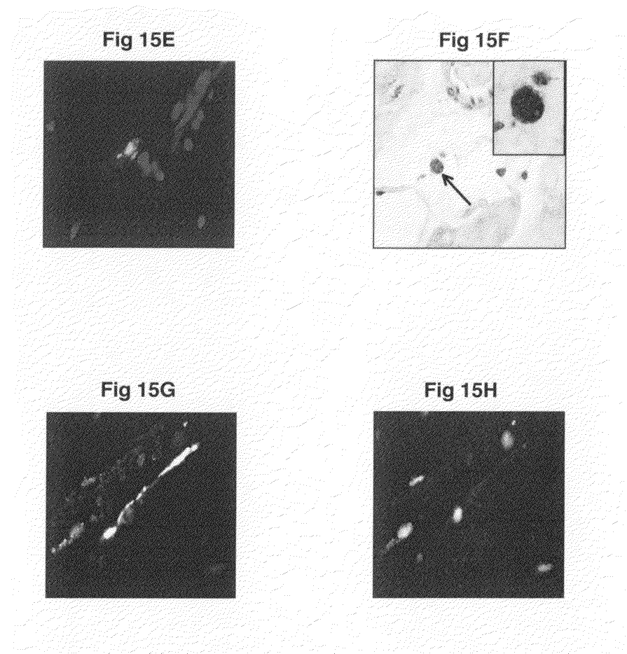

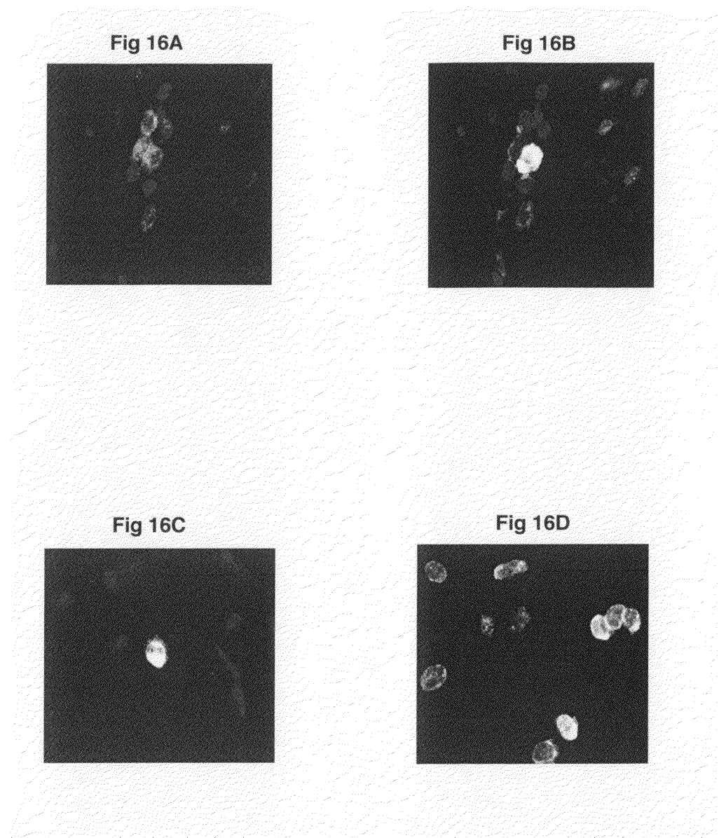

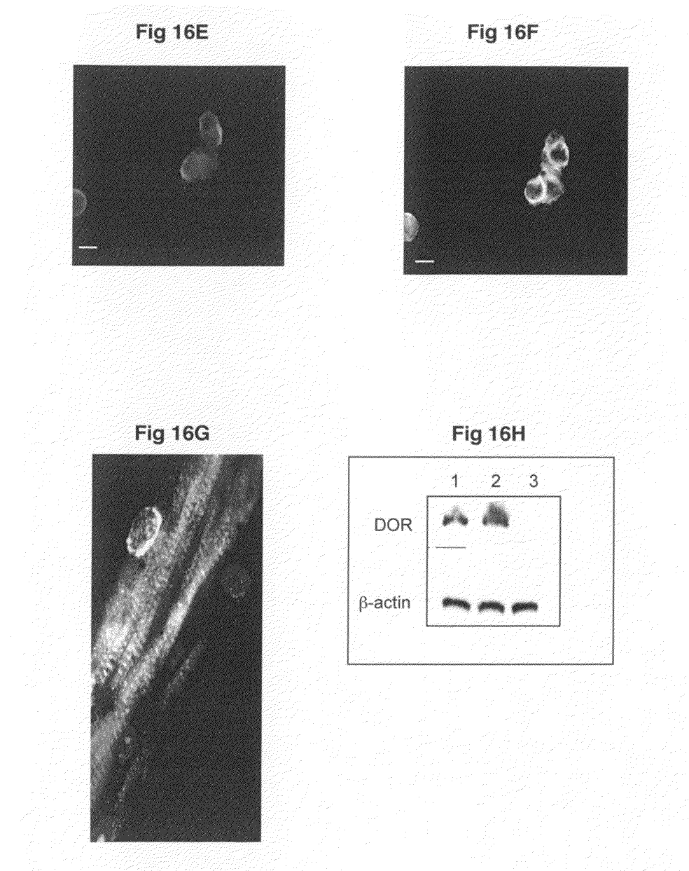

The d-opioid receptor immunoreactivity was exclusively co-localized with tyrosine hydroxylase in human and adult rat intrinsic cardiac adrenergic cells (FIGS. 15 and 16). Ventricular myocytes but intrinsic cardiac adrenergic cells express myosin heavy chain immunoreactivity (FIG. 16G). Over 90% of magnetically isolated cardiocytes cells express tyrosine hydroxylase and d-opioid receptor immunoreactivity yielding extremely high intrinsic cardiac adrenergic cell purity (FIG. 16D). d-opioid receptor-expressing intrinsic cardiac adrenergic cells constitute ˜13% of total cardiocytes based on the cell counting of dissociated rat cardiocyte preparation. No d-opioid receptor immunoreactivity was identified in human ventricular myocytes in tissue sections (FIG. 16G) or isolated rat ventricular myocytes (FIG. 16C), or sympathetic nerve endings (FIG. 15H). No immunoreactivity was detected in IgG control slides. The control slides for double-labeling that was stained with only Streptavidin-ALEXA FLUOR® 448 after step 2 showed no cross-reactivity to d-opioid receptor confirming immunohistochemical specificity of d-opioid receptor expression in intrinsic cardiac adrenergic cells. For in situ hybridization intrinsic cardiac adrenergic cells were not labeled with tyrosine hydroxylase oligonucleotide sense probe. Western blot analysis detected DOR protein band in rat intrinsic cardiac adrenergic cell isolates, which is identical to that found in rat brain tissue. No d-opioid receptor protein was detected in rat ventricular myocytes with depleted intrinsic cardiac adrenergic cells (FIG. 16H).

### EXAMPLE 16

### Modulation of [Ca2+]I Transients in Rat Intrinsic Cardiac Adrenergic Cells

The effects of a potent δ-opioid agonist [D-Pen²⁵]-enkephalin (DPDPE) were studied on [Ca²⁺]i transients generated by isolated intrinsic cardiac adrenergic cells in culture. After obtaining baseline [Ca²⁺]i transients of an intrinsic cardiac adrenergic cell for 15 min, [D-Pen²⁵]-enkephalin at different doses (0.1 nmol/L, 1 nM, 10 nM and 100 nmol/L) was administrated in random order to the intrinsic cardiac adrenergic cell for 15 min, respectively. The intrinsic cardiac adrenergic cells were washed for 20 min, while continuously recording [Ca²⁺]i transients until the activity reached a plateau. The specificity of [D-Pen²⁵]-enkephalin was examined by exposing intrinsic cardiac adrenergic cells to the d-opioid receptor antagonist naltrindole (1 mmol/L) for 10 min followed by application of [D-Pen²⁵]-enkephalin (100 nmol/L) and naltrindole (1 mmol/L) for 15 min. The involvement of adenylyl cyclase-cAMP-protein kinase A in d-opioid receptor signaling was examined by application of [D-Pen²⁵]-enkephalin (100 nmol/L for 15 min) to intrinsic cardiac adrenergic cells that had been pretreated with a protein kinase A inhibitor Rp-CAMPS (100 μmol/L) for 30 min to block protein kinase A activity. To determine whether the L-type Ca²⁺ channels were responsible for altered [Ca²⁺]i transients following d-opioid receptor stimulation, the effects of [D-Pen²⁵]-enkephalin on intrinsic cardiac adrenergic cells in the presence of the L-type Ca²⁺ channel blocker nifedipine was tested. Intrinsic cardiac adrenergic cells were perfused with nifedipine (1 mmol/L) for 5 min following by administration of nifedipine plus [D-Pen²⁵]-enkephalin (10 nmol/L) for 15 min.

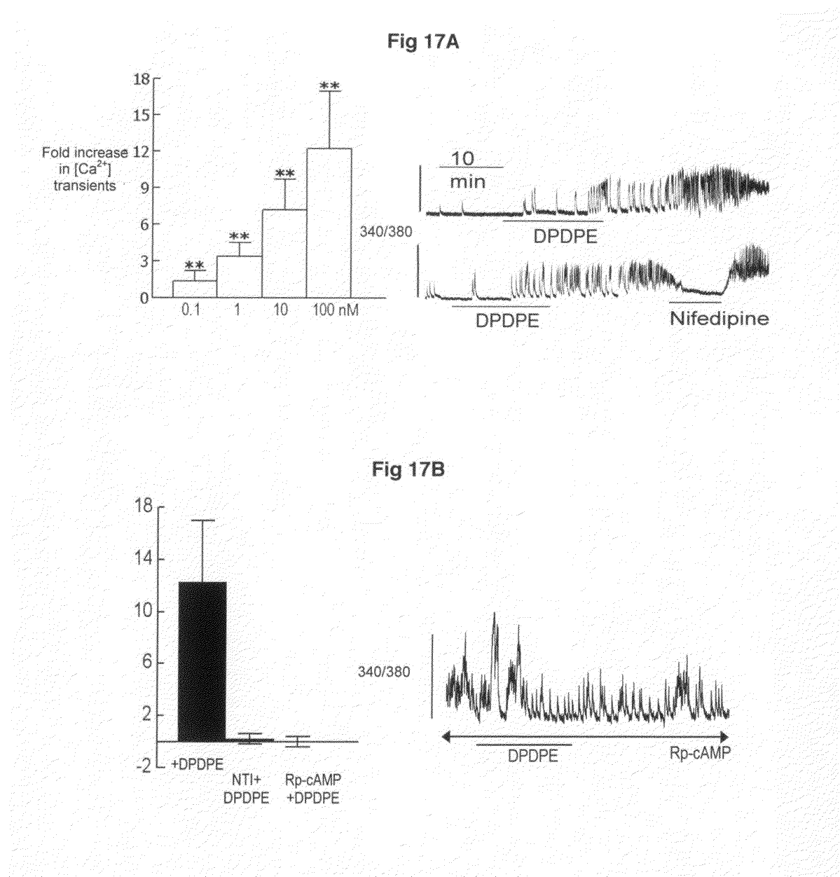

Application of [D-Pen²⁵]-enkephalin to intrinsic cardiac adrenergic cells increased [Ca²⁺]i transient spikes in a concentration-dependent manner (FIG. 17). Increases in [Ca²⁺i transients were sometimes preceded by a brief quiescent (inhibitory period) phase. [D-Pen²⁵]-enkephalin did not change [Ca²⁺]i transient activity significantly in the presence of the DOR antagonist natrindole (1 mmol/L) (FIG. 17). The excitatory effect of [D-Pen²⁵]-enkephalin on [Ca²⁺]i transients of intrinsic cardiac adrenergic cells was completely abolished following the application of nifedipine (1 mmol/L, n=3). Following treatment of intrinsic cardiac adrenergic cells with Rp-cAMPS (100 μmol/L for 30 min), application of [D-Pen²⁵]-enkephalin (100 nmol/L, n=5) no longer modified [Ca²⁺]i transients (FIG. 17B).

### EXAMPLE 17

d-Opioid Receptor-Induced Epinephrine Release

After 3 days in culture, the medium of intrinsic cardiac adrenergic cells was removed and the intrinsic cardiac adrenergic cells were incubated with 500 μL of Tyrode's solution for 1 hr. The sample solution was collected for basal catecholamine release. Then the culture wells were treated for 30 min with 500 μl Tyrode's solution with or without [D-Pen²⁵]-enkephalin (100 nmol/L). Conditioned solutions were collected at the end of 30 min and the culture wells were washed 3 times. After washing, 500 μl of Tyrode's solution was reintroduced to the culture wells for 1 hr (recovery phase). At the end of the recovery phase, the samples were collected. The cells were lysed and their protein contents quantified to standardize catecholamine release.

Catecholamine release from isolated intrinsic cardiac adrenergic cells in culture was determined using a high-performance liquid chromatography system (Cui et al, 1999). The detection limit was determined by making multiple injections of diluted standards into the column. Peak areas are a linear function of the amount of substance injected over the concentration range of the samples. An external standard was run every five samples. Data were collected and analyzed with CHROMGRAPH® software (Bioanalytical System, West Lafayette, Ind.).

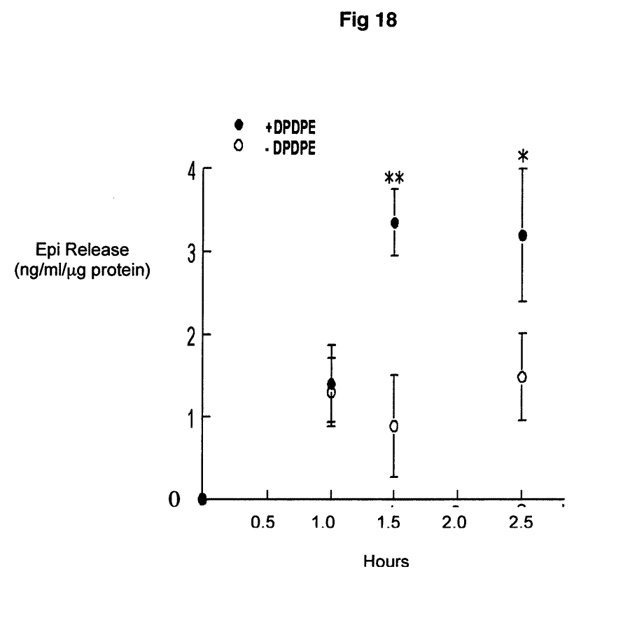

Basal epinephrine release from intrinsic cardiac adrenergic cells was detected in culture. Application of [D-Pen²⁵]-enkephalin (100 nmol/L) increased epinephrine release 2.4-fold. Application of Tyrode's solution to intrinsic cardiac adrenergic cells did not affect epinephrine release (FIG. 18). Norepinephrine was not consistently detected either at baseline, during [D-Pen²⁵]-enkephalin treatment, or during the recovery phase, presumably due to insignificant release below the detection limit of the high-performance liquid chromatography system.

### EXAMPLE 18

### DOR-Induced Infarct Size Reduction

The rat myocardial infarct model has been described in detail (22). To demonstrate δ-opioid agonist-initiated infarct size reduction, [D-Pen²⁵]-enkephalin at an effective dose of 200 ng/kg (16) was infused intravenously 30 min before the coronary artery occlusion. To determine whether the infarct-limiting effect of [D-Pen²⁵]-enkephalin is dependent on endogenous catecholamine, the combined α- and beta-adrenergic receptor blocker labetalol (2 mg/kg) was infused intravenously over 30 min followed by administration of [D-Pen²⁵]-enkephalin at 30 min before the coronary artery occlusion. For the control group saline was infused (n=6 for each group).

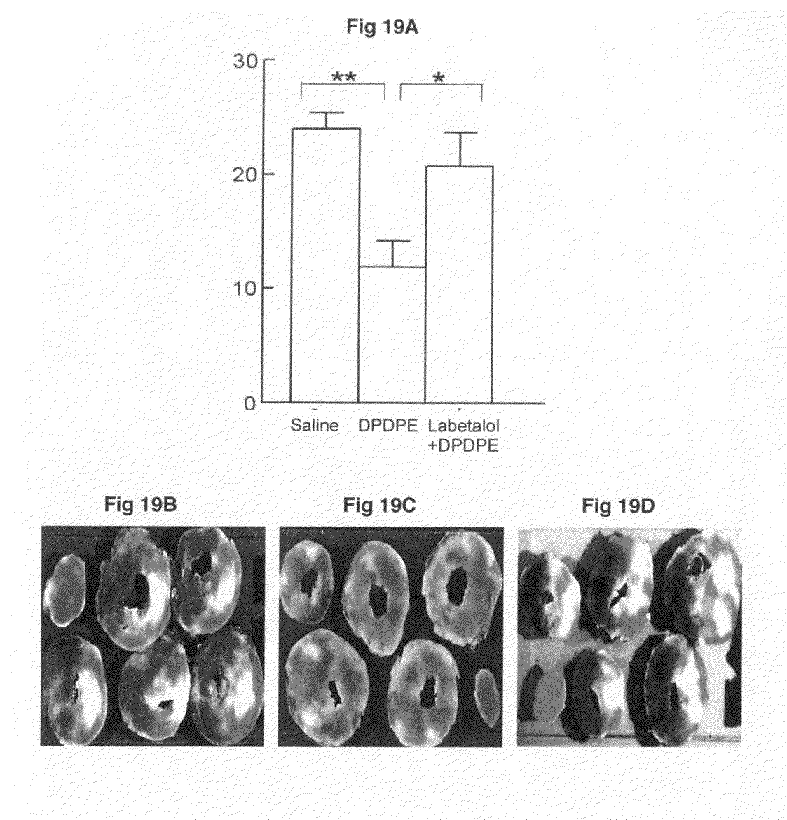

Infusion of [D-Pen²⁵]-enkephalin prior to coronary artery occlusion reduces LV infarct size by 53% compared to control. This infarct-size-limiting effect by [D-Pen²⁵]-enkephalin was nearly abolished when the rats had been pretreated with labetalol (FIG. 19). There is no significant difference in the body weight, LV weight and area at risk among three animal groups (Table 1A). There is a small reduction in the mean blood pressure in the animal group that received labetalol compared to the others indicative of effectiveness of vascular α-adrenergic receptor blockade. There is mild increase in the heart rate in the animal group that received labetalol (Table 1B).

TABLE 1
Infarct size among three animal groups. 1B: Mean BP and HR during
experimental myocardial infarction among three animal groups.

| N = 6 | Control | Labetalol | DPDPE | Labetalol + DEDPE | P value |
|---|---|---|---|---|---|
| Body wt (g) | 288.5 ± 6.0 | 286 ± 5.1 | 287.5 ± 4.1 | 289.7 ± 4.7 | ns |
| LV wt (mg) | 800 ± 1 | 801 ± 1 | 800 ± 1 | 800 ± 1 | ns |
| Area at Risk (% of LV) | 52.4 ± 1.5 | 58.5 ± 1.8 | 54.6 ± 1.3 | 55.8 ± 0.7 | ns |
| Infarct Size (% of LV) | 13.3 ± 1.1 | 16.2 ± 1.7 | 6.6 ± 1.4 | 11.6 ± 1.5 | <0.01 |

(1B) Hemodynamic changes in 3 experimental infarct animal groups

| | | | 25 min | 20 min |
|---|---|---|---|---|
| | Baseline | Pre-Occlusion | Occlusion | Reperfusion |
| Mean BP (mmHg) | | | | |
| Control | 123 ± 1 | 115 ± 1 | 90 ± 1 | 95 ± 1 |
| Labetalol | 125 ± 2 | 100 ± 2* | 78 ± 4** | 81 ± 4** |
| DPDPE | 125 ± 1 | 116 ± 1 | 89 ± 2 | 96 ± 1 |
| Labetalol + | 126 ± 1 | 106 ± 3* | 82 ± 2** | 87 ± 2** |
| DPDPE | | | | |
| HR (bpm) | | | | |
| Control | 216 ± 1 | 219 ± 2 | 229 ± 2 | 215 ± 1 |
| Labetalol | 213 ± 1 | 223 ± 1* | 244 ± 2 | 235 ± 2** |
| DPDPE | 215 ± 1 | 218 ± 1 | 232 ± 1 | 216 ± 1 |
| Labetalol + | 215 ± 1 | 224 ± 2* | 233 ± 2 | 226 ± 1** |
| DPDPE | | | | |

### EXAMPLE 19

### Data Analysis

The spike frequency of intrinsic cardiac adrenergic cell [Ca²⁺]I transients is analyzed for each intervention. Baseline and steady state peak response (5 min) are compared using Student's t-test or ANOVA. ANOVA is also used to analyze brain natriuretic peptide release from intrinsic cardiac adrenergic cells in response to [D-Pen²⁵]-enkephalin treatment.

Data are presented as mean±SE. The significance level α is 0.05. For quantification of [Ca²⁺]i transients generated by intrinsic cardiac adrenergic cells, the firing frequency of [Ca²⁺]i transients generated by intrinsic cardiac adrenergic cells was determined. For quiescent cells, an average of 5 min duration was used. The outcome measure was the number of spikes/min in each phase. ANOVA and ANOVA with two-way repeated-measures were used for analyzing the changes in [Ca²⁺]i transient dynamics, catecholamine release, infarct sizes, and differences in the heart rate and mean blood pressure between the animal groups.

### EXAMPLE 20

### Brain Natriuretic Peptide is Up-Regulated in Intrinsic Cardiac Adrenergic Cells

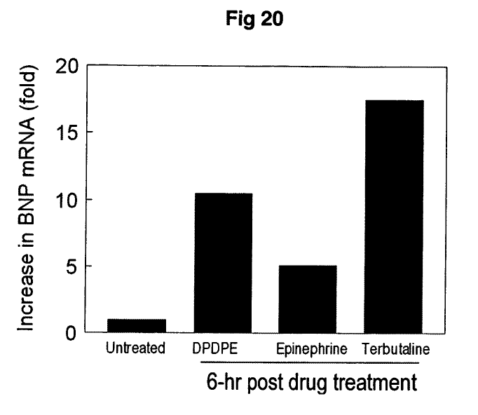

In isolated rat intrinsic cardiac adrenergic cell culture, we have demonstrated that brain natriuretic peptide gene can be up-regulated by the treatment of the d-opioid receptor agonist DPDPE, selective b2-adrenergic receptor agonist terbutaline, or non-selective b-adrenergic receptor agonist epinephrine (FIG. 20). Thus, using d-opioid receptor agonist or b2-adrenergic receptor agonists may provide effective strategies for the congestive heart failure treatment via mobilization of endogenous brain natriuretic peptide production in intrinsic cardiac adrenergic cells (i.e., brain natriuretic peptide mRNA upregulation).

The following references are cited herein:
1. VMAC Investigators. JAMA 287:1531-1540, 2002.
2. De Lemos J A, Morrow D A. Circulation 106:2868-2870, 2002.
3. Maisel et al. N. Engl. J. Med. 347:161-167, 2002.
4. Colucci et al. N. Engl. J. Med. 343:246-253, 2000.
5. Clarkson P B M., Circulation 93:2037-2042, 1996.
6. Lang C c, Motwani j G, Coultie W, et al. Lancet 338:255, 1991.
7. Kentsch M, Otter W. Drugs RD 1:331-338, 1999.
8. Huang et al., J Clin Invest. 98:1298-1303, 1996.
9. Huang et al., Circulation 108 (Suppl iV) IV-871, 2003.
10. Bankwala Z et al., Circulation 90:1023-1028, 1994.
11. Cleveland J C et al., Am J Physiol. 273:H902-908, 1997.
12. Kitakaze M et al., J Clin Invest. 93:2197-2205, 1994.
13. Bell S P et al., J Am Coll Cardiol 36: 2296-302, 2000.
14. Guo Y et al., Am J Physiol-Heart Circ Physiol. 289:H2251-2257, 2005
15. Schultz J E et al., Circulation. 97:1282-1289, 1998.
16. Sigg D et al. Am J Physiol-Heart Circ Physiol. 282:H1963-H1960, 2002.
17. Fryer R M et al., Circ Res. 84:846-851, 1998.
18. Fryer R M et al., Am J Physiol-Heart Circ Physiol. 280:H1346-1353, 2001.
19. Ito K et al., J Physiol. 532:165-174, 2001.
20. Kaku T et al., Am J Physiol. 260: C635-642, 1991.
21. Huang M H et al., Am J Physiol Heart-Circ Physiol. 288:H497-H503, 2005.
22. Birnbaum Y et al., Cardiovasc Res. 65:345-355, 2005.
23. Cui M et al., J Pharmacol Exp Ther. 289-868-876, 1999.

What is claimed is:

1. A method of treating an individual having a cardiovascular disease, comprising administering to said individual a pharmacologically effective dose of a compound that stimulates release of endogenous brain natriuretic peptide from intrinsic cardiac adrenergic cells.

2. The method of claim 1, wherein said cardiovascular disease is decompensated congestive heart failure, or myocardial ischemic disease.

3. The method of claim 1, wherein said compound stimulates δ-opioid receptors or selective β2-adrenergic receptors in said intrinsic cardiac adrenergic cells to initiate synthesis and release of said brain natriuretic peptide.

4. The method of claim 1, wherein said compound is administered via an oral, an intramuscular, an intravenous, an intradermal, or a subcutaneous route.

5. The method of claim 1, further comprising the step of administering exogenous brain natriuretic peptide in said individual.

6. A method of treating an individual suffering from a cardiovascular disease, comprising: administering to said individual a pharmacologically effective dose of a compound that up-regulates catecholamine synthesis and release from intrinsic cardiac adrenergic cells.

7. The method of claim 6, wherein said compound stimulates δ-opioid receptors or selective β2-adrenergic receptors in said intrinsic cardiac adrenergic cells to up-regulate synthesis and release of said catecholamines.

8. The method of claim 6, wherein said catecholamine is selected from a group consisting of norepinephrine and epinephrine.

9. The method of claim 6, wherein said compound is [D-Pen²⁵]-enkephalin, a deltorphin, terbutaline, or D-Ala2-D-Leu5-enkephalin.

10. The method of claim 6, wherein said disease is myocardial ischemia, or decompensated congestive heart failure.

11. The method of claim 6, wherein said compound activates protein kinase A.

12. The method of claim 6, wherein said compound generates [Ca²⁺]ᵢ transients required for release of said catecholamines.

13. The method of claim 6, further comprises the step of administering exogenous synthetic catecholamines to said individual.

14. The method of claim 13, wherein said catecholamines is selected from a group consisting of dobutamine, dopamine, norepinephrine, terbutaline, and epinephrine.

15. The method of claim 6, wherein said compound is administered via oral, intramuscular, intravenous, intradermal, or subcutaneous route.

16. A method of treating an individual suffering from a cardiovascular disease, comprising: administering to said individual a pharmacologically effective dose of a compound that inhibits catecholamine synthesis and release from intrinsic cardiac adrenergic cells wherein the heart of said individual is protected from excessive beta-adrenergic stimulation.

17. The method of claim 16, wherein said compound stimulates δ-opioid receptors in said intrinsic cardiac adrenergic cells to down-regulate synthesis and release of said catecholamines.

18. The method of claim 16, wherein said heart disease is myocardial ischemia, or decompensated congestive heart failure.

19. The method of claim 16, wherein said compound is a calcium channel blocker.

20. The method of claim 19, wherein said calcium channel blocker is nifedipine.

21. The method of claim 16, wherein said compound inhibits activation of protein kinase A.

22. A method of treating an individual with a disease characterized by excessive fluid retention, comprising: administering to said individual a pharmacologically effective dose of a compound that stimulates release of brain natriuretic peptide to produce diuresis.

23. The method of claim 22, wherein said disease is cirrhosis.

24. The method of claim 22, wherein said compound stimulates δ-opioid receptors or selective β2-adrenergic receptors to initiate synthesis and release of said brain natriuretic peptide.

25. The method of claim 22, wherein said compound is administered via an oral, intramuscular, intravenous, intradermal, or subcutaneous route.

26. A method of stimulating release of brain natriuretic peptide from intrinsic cardiac adrenergic cells in an individual in need of such treatment, comprising: administering pharmacologically effective dose of a δ-opioid receptor agonist or a selective β2-adrenergic receptor agonist to said individual.

27. The method of claim 26, wherein said individual has myocardial ischemia, or decompensated congestive heart failure.

28. The method of claim 26, wherein said δ-opioid receptor agonist or said selective β2-adrenergic receptor agonist are administered via an oral, an intramuscular, an intravenous, an intradermal, or a subcutaneous route.

29. A method of inducing cardioprotection in an individual in need of such treatment comprising: administering pharmacologically effective dose of a compound that activates intrinsic cardiac adrenergic cells.

30. The method of claim 29, wherein said compound induces epinephrine release from said intrinsic cardiac adrenergic cells, is an agonist of one or more than one δ-opioid receptors or selective β2-adrenergic receptors in said intrinsic cardiac adrenergic cells, generates [Ca²⁺]ᵢ transients in said intrinsic cardiac adrenergic cells, activates protein kinase A in said intrinsic cardiac adrenergic cells, or a combination thereof.

31. The method of claim 29, wherein said agonist is [D-Pen²⁵]-enkephalin, a deltorphin, terbutaline, or D-Ala2-D-Leu5-enkephalin.

32. The method of claim 29, wherein said compound is administered via oral, intramuscular, intravenous, intradermal or subcutaneous route.

33. The method of claim 29, wherein the said individual has myocardial ischemia or decompensated congestive heart failure.


SEQUENCE LISTING (6 sequences)

SEQ ID NO: 1 - DNA, 22 bp, Artificial sequence
  forward primer for tyrosine hydroxylase
aactctccacggtgtactggtt

SEQ ID NO: 2 - DNA, 22 bp, Artificial sequence
  reverse primer for tyrosine hydroxylase
gcatagttcctgagcttgtcct

SEQ ID NO: 3 - DNA, 22 bp, Artificial sequence
  reverse primer for phenylethanolamine-N-methyl transferase
actggagtgtgtatagccagca

SEQ ID NO: 4 - DNA, 22 bp, Artificial sequence
  reverse primer for phenylethanolamine-N-methyl transferase
acactggaaccacagatagcct

SEQ ID NO: 5 - DNA, 22 bp, Artificial sequence
  forward primer for brain natriuretic peptide
tgacgggctgaggttgttttag

SEQ ID NO: 6 - DNA, 24 bp, Artificial sequence
  reverse primer for brain natriuretic peptide
ggcaagtttgtgctggaagataag